(19) United States
(12) Patent Application Publication
(10) Pub. No.: US 2016/0216244 A1
(43) Pub. Date: Jul. 28, 2016

(54) METHOD AND ELECTRONIC NOSE FOR COMPARING ODORS

(71) Applicant: YEDA RESEARCH AND DEVELOPMENT CO. LTD., Rehovot (IL)

(72) Inventors: Noam SOBEL, Jaffa (IL); Kobi SNITZ, Rehovot (IL); Adi YABLONKA-BARAK, Rehovot (IL); Tali WEISS, Rehovot (IL); Idan FRUMIN, Rehovot (IL); Aharon RAVIA, Rehovot (IL)

(73) Assignee: Yeda Research and Development Co. Ltd., Rehovot (IL)

(21) Appl. No.: 14/917,290

(22) PCT Filed: Sep. 11, 2014
(86) PCT No.: PCT/IL2014/050812

§ 371 Date: Mar. 8, 2016

Related U.S. Application Data
(60) Provisional application No. 61/876,785, filed on Sep. 12, 2013.

(51) Int. Cl. G01N 33/00

(52) U.S. Cl. CPC G01N 33/0062; G01N 2033/0068

(57) ABSTRACT

A method for comparing odors comprises: sampling odor sources and detecting primary odorants, then for each odor source, storing each of the sampled odor sources in respective primary vectors of odor descriptors that describe the primary odorants. For each source a source vector is then constructed by summing the primary vectors of the respectively detected primary odorants. Comparison between the odors is achieved by determining an angle between the source vectors, which may then be output. The method may be used in electronic noses and like equipment.

### RELATED APPLICATION/S

This application claims the benefit of priority under 35 USC §119(e) of U.S. Provisional Patent Application No. 61/876,785 filed Sep. 12, 2013, the contents of which are incorporated herein by reference in its entirety.

### FIELD AND BACKGROUND OF THE INVENTION

The present invention, in some embodiments thereof, relates to a method and apparatus for predicting odor perceptual similarity from odor structure.

One hundred years ago, Alexander Graham Bell asked: “Can you measure the difference between one kind of smell and another. It is clear that we have very many different kinds of smells, ranging from the odor of violets and roses on the pleasant side to asafoetida at the unpleasant end. But until you can measure their likenesses and differences you can have no science of odor.”. Although the challenge posed by Bell has been widely recognized in olfaction research, the field has yet to gravitate to an agreed upon system for odor measurement.

Early investigations into quantification of odor revolved around an effort to identify odor primaries, similar to the notion of primary colors in vision. A major tool in this effort was the quantification of specific anosmias. Although specific anosmia remains a powerful tool for linking odor perception to olfactory neurobiology, this path did not generate a general method to quantify olfactory perception. A conceptually similar approach was an effort to identify specific odorant molecular features that drove specific olfactory perceptual notes. This approach, referred to as structure-odor-relationships or SOR, identified many specific rules linking structure to odor (e.g., what structure provides a “woody” note), but failed to produce a general framework for measuring smell.

An alternative path to measuring smell was to identify general perceptual primaries rather than individual odorant primaries. This approach, consisting of applying statistical dimensionality reduction to many perceptual descriptors applied to many odorants, repeatedly identified odorant pleasantness, namely an axis ranging from very unpleasant to very pleasant, as the primary dimension in human olfactory perception. Initial efforts to link such perceptual axes to odorant structural axes saw only limited success because of the limited scope of physicochemical features one could easily obtain for a given molecule. However, the recent advent of software that provides thousands of physicochemical descriptors for any molecule (e.g. Dragon 5™ and Dragon 6™ produced by Talete s.r.l. of Milan, Italy) allows application of similar dimensionality reduction to odorant structure as well. This process reveals odorant structural dimensions that are modestly but significantly predictive of odorant perception and odorant-induced neural activity across species.

Although the above studies combine to generate an initial form of olfactory metrics, they all apply to mono-molecular odorants alone. The real olfactory world, however, is not made of mono-molecules, but rather of complex olfactory multi-molecular mixtures. For example, roasted coffee, red wine, or rose, each contain hundreds of different mono-molecular species, many of them volatile. Thus, a useful metric for smell must apply to such odorant-mixtures.

### SUMMARY OF THE INVENTION

The present embodiments compare smells of multi-molecular mixtures using a model that represents each mixture as a single structural vector.

Olfactory processing of stimuli with given physicochemical properties begins with sensing it and ends in producing a certain percept. The ability to predict the percept of a stimulus from its physicochemical properties may provide a tool in studying the process of perception. A first step towards such a tool is identifying a way to measure how close or far different percepts are. Herein, the ‘perceptual distance’ between odorants defines similarity ratings given by human subjects, and that distance is related to the differences in physicochemical properties of the stimuli.

Since most naturally occurring odorants are mixtures of molecules, the present embodiments focus on the properties of odor mixtures. This presents a preliminary question which has clear biological implications: is a mixture perceived as a collection of components or as a unified percept? It is shown herein that a unified percept model outperforms a model based on representing odorants as collections of components. This is especially notable since the unified percept model is based on much less information. A model according to the present embodiments was tested on mono-molecules and different sizes of mixtures from three separate experiments and may be shown to work consistently under different conditions. This forms a useful link between description of stimuli and their percepts. With it one can now see the effect of a measured change in perception on neuronal activation etc.

According to an aspect of some embodiments of the present invention there is provided a method for comparing odors comprising:

sampling a first odor source and detecting primary odorants of said first odor source;

sampling a second odor source and detecting primary odorants of said second source;

for each odor source, storing each of the sampled odor sources in respective primary vectors of odor descriptors;

for each source respectively building a source vector of detected primary odorants by summing said primary vectors of the respectively detected primary odorants;

determining an angle between said first and second source vectors; and

outputting said determined angle as a comparison between said first and second odor sources.

An embodiment may comprise determining said angle from a dot product calculated between said source vectors.

An embodiment may comprise determining said angle by normalizing said dot product, said normalizing comprising dividing said dot product by a multiple of norms of said source vectors to obtain a normalized ratio.

An embodiment may comprise obtaining said angle by applying an inverse cosine operation to said normalized ratio.

In an embodiment, said descriptors making up said primary vectors are constructed from a set of physicochemical odor descriptors.

Dimension reduction may be carried out to get a reasonable sized set of descriptors. The dimension reduction may involve a two-stage bootstrapping process, of which the first stage may comprise obtaining an initially relatively large set of said physicochemical descriptors and carrying out dimension reduction by retaining ones of said of physicochemical descriptors shown experimentally to contribute by more than an average to a final comparison result.

In an embodiment, said initially relatively large set comprises is in excess of a thousand of said of physicochemical descriptors of which a set of twenty is retained following said dimension reduction, such that said component vectors have a dimension of twenty.

An embodiment may carry out normalizing the respective source vectors.

A device for detecting primary odorants may be based on a GCMS or an electronic nose device for detecting and comparing odors, and may comprise: a sampling unit configured to sample odor sources and detect primary odorants therein;

a vectorising unit for configured to store each of the sampled odor sources as respective primary vectors, the primary vectors each defining one of said detected primary odorants in terms of a predetermined set of odor descriptors;

a summation unit configured to build a source vector for each detected odor source by summing said respective primary vectors and normalizing;

an odor comparison unit, configured to compare two detected odor sources by determining an angle between respective source vectors.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

According to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified flow chart illustrating a first embodiment of a process for distinguishing odors according to the present invention;

FIG. 2 is a simplified flow chart showing in greater detail the determination of an angle of the embodiment of FIG. 1;

FIG. 3 is a simplified block diagram illustrating an electronic nose according to an embodiment of the present invention;

FIGS. 4A and 4B show odorants plotted over a perceptual and physic-chemical spaces respectively;

FIG. 4C schematically illustrates comparisons made between different odor mixtures;

FIGS. 5A and 5B show side by side comparisons of a model comparing odor components directly, and a model using a single vector representation according to the present embodiments;

FIGS. 6A and 6B are graphs showing mean pairwise distance against rated similarity for two experiments and showing little correlation.

FIGS. 6C and 6D are graphs showing the angle distance model using a single vector representation according to the present embodiments, and achieving some correlation;

FIG. 7A is a simplified graph showing the effect of a number of features in the feature space on the correlation level of the overall source vector;

FIG. 7B is a simplified graph showing the effects of individual features in the feature space on the correlation level of the overall source vector, and showing clearly that certain descriptors are of particular importance, allowing construction of a reduced dimension set of descriptors according to embodiments of the present invention;

FIG. 8 is a graph showing the angle distance model using a single vector representation according to the present embodiments including the optimizations, and achieving a clear correlation;

FIG. 9A is a graph illustrating performance of the optimized model on complete Dataset #1, and wherein each dot reflects a comparison between two mixtures;

FIG. 9B is a graph of the same data as in FIG. 9A after omitting comparisons of mixtures to themselves;

FIG. 9C is an RMSE histogram reflecting the performance of random selections of 21 descriptors;

FIG. 9D shows performance of the optimized angle distance model on the mono-molecules of Dataset #3;

FIG. 9E illustrates performance of the angle distance model on mono-molecules tested 50 years ago independently by others;

FIG. 9F illustrates performance of the optimized angle distance model on the data in FIG. 9E, and wherein each dot reflects a comparison between two mono-molecules;

FIG. 10 is a graph predicting the presence of Olfactory White based on the number of components using the angle distance model;

FIG. 11 is a graph showing mean pairwise distances plotted against average rated similarity for experiment A and showing no correlation;

FIG. 12 is the dataset of FIG. 11 with identical comparisons removed;

FIG. 13 is a graph showing the number of descriptors as a function of mean error in comparisons of the odors;

FIG. 14 illustrates contributions of individual descriptors to the overall comparison result;

FIG. 15 is a graph illustrating the performance of a set of 21 best descriptors selected according to the two-stage training process and FIG. 14, when tested on a testing set and showing results of RMSE=6.98 r=−0.85 p<0.001;

FIG. 16 is a graph obtained using the same experiment as in FIG. 15 but carried out on different data;

FIG. 17 is an RMSE histogram, showing error ranges for the optimized and other randomly selected sets of 21 descriptors; and

FIG. 18 is a graph showing angular distance against average rated similarity for the mono molecules of all data sets taken together.

### DESCRIPTION OF SPECIFIC EMBODIMENTS OF THE INVENTION

The present invention, in some embodiments thereof, relates to a method and apparatus for predicting perceptual odor similarity from molecular structure and, more particularly, but not exclusively, to odor similarity of complex olfactory multi-molecular mixtures.

A method for comparing odors comprises: sampling odor sources and detecting primary odorants, then for each odor source, storing each of the sampled odor sources in respective primary vectors of odor descriptors that describe the primary odorants. For each source, a source vector is then constructed by summing the primary vectors of the respectively detected primary odorants. Comparison between the odors is achieved by determining an angle between the source vectors, which may then be output. The method may be used in electronic noses and like equipment, and has application in food preparation and storage, as well as detection of contraband, search and rescue operations and many other fields where smell needs to be measured.

The present embodiments provide a way of comparing complex olfactory multi-molecular mixtures smell to each other in a way that predicts their perceptual similarity. The present inventors collected perceptual similarity estimates from a large group of subjects rating a large group of odorant-mixtures of known components. Subsequently the present inventors tested alternative models linking odorant-mixture structure to odorant-mixture perceptual similarity, and have thus provided a device and method that provides a meaningful predictive framework for odor comparison. Using the method it is possible to look at novel mono-molecular odorants, or multi-component odorant-mixtures, and predict their ensuing perceptual similarity.

To understand the brain mechanisms of olfaction one must understand the rules that govern the link between odorant structure and odorant perception. Natural odors are in fact mixtures made of many molecules, and there is currently no method to look at the molecular structure of such odorant-mixtures and predict their smell.

As described below, in three separate experiments, the present inventors ask 139 subjects to rate the pairwise perceptual similarity of 64 odorant-mixtures ranging in size from 4 to 43 mono-molecular components. The present inventors then test alternative models to link odorant-mixture structure to odorant-mixture perceptual similarity. Whereas a model that considers each mono-molecular component of a mixture separately provides a poor prediction of mixture similarity, a model that represents the mixture as a single structural vector provides consistent correlations between predicted and actual perceptual similarity (r=0.49, p<0.001). An optimized version of the single structure model yields a correlation of r=0.85 (p<0.001) between predicted and actual mixture similarity. The present embodiments thus make use of an algorithm that can look at the molecular structure of two novel odorant-mixtures, and predict their ensuing perceptual similarity. That this goal was attained using a model that considers the mixtures as a single vector is consistent with a synthetic rather than analytical brain processing mechanism in olfaction.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

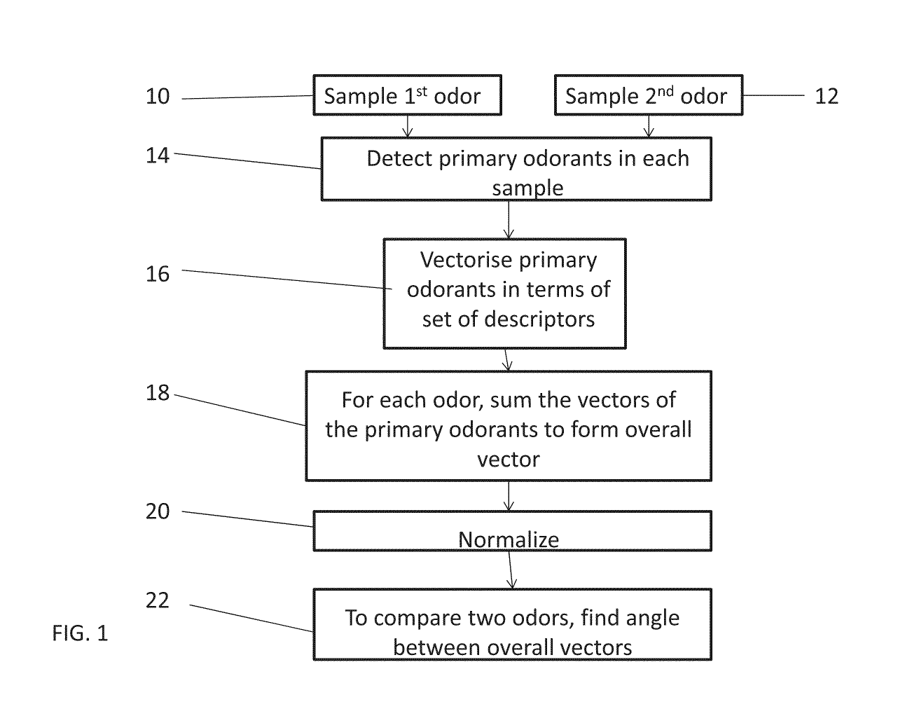

Referring now to the drawings, FIG. 1 is a simplified flow chart that illustrates a method for comparing odors according to an embodiment of the present invention. The two odors to be compared are initially sampled 10, 12, and primary odorants are identified or detected 14. A closed set of odor descriptors characterizes each primary odorant, and thus each primary odorant can be vectorized 16 in terms of the set of primary odorants. Thus each of the sample odors is at this stage recorded as a series of individual or primary vectors.

Vectors are then built 18 describing the overall odor. For each odor source a source vector is generated simply by summing the corresponding primary vectors. All the vectors are of the same dimension since they all rely on the same set of descriptors, so that summation is a defined operation. The vectors may need to be normalized 20 if different odors have different numbers of primary odorants.

Then, in order to compare two odors, the source vectors are compared 22 by determining the angle between the vectors. As the source vectors are of the same dimension, the dot product is a fully defined operation between the normalized vectors. Using the dot product, an angle is determined between the source vectors, which can be output as a difference between the odors.

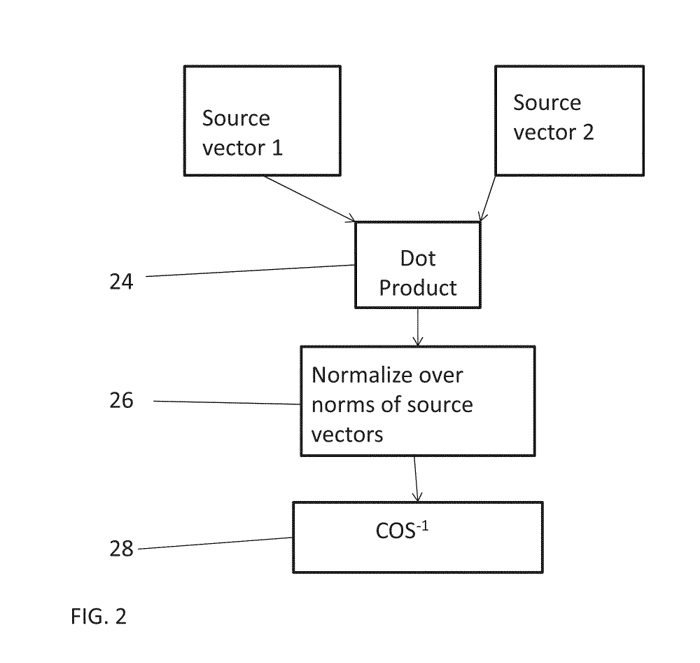

Reference is now made to FIG. 2, which shows in greater detail the process of comparing the angles of the two source vectors of FIG. 1. The two source vectors to be compared, source vector 1 and source vector 2 are combined by forming the dot product 24. The dot product result is normalized 26 over the product of the norms of the two source vectors and then the inverse cosine is calculated, to produce the actual comparison angle.

The descriptors used may be a set of physicochemical odor descriptors. As will be explained in greater detail below, initially a set of descriptors covering as much as possible of smell space is selected. Unfortunately, however this may be a very large number of descriptors and lead to a very large dimensional problem, with vectors having some one and a half thousand dimensions. Thus dimension reduction of the descriptors may be carried out to produce a more manageable set of descriptors. As will be discussed in greater detail below, experimental work combined with statistical operations may be used to identify a reduced list of around twenty descriptors without losing much in the way of resolution.

Thus, dimension reduction may involve a two stage bootstrapping process to reduce the dimension of the odorant descriptors from about 1500 to about 20, the first stage of which comprises arranging sets of descriptors and then removing one descriptor to find out what difference results. Eventually the descriptors which contribute by more than an average to a final comparison result are retained.

Assuming a set of twenty descriptors, both the primary vectors and the source vectors may have a dimension of twenty, allowing summation and dot product operations to be carried out with ease on modern computing devices.

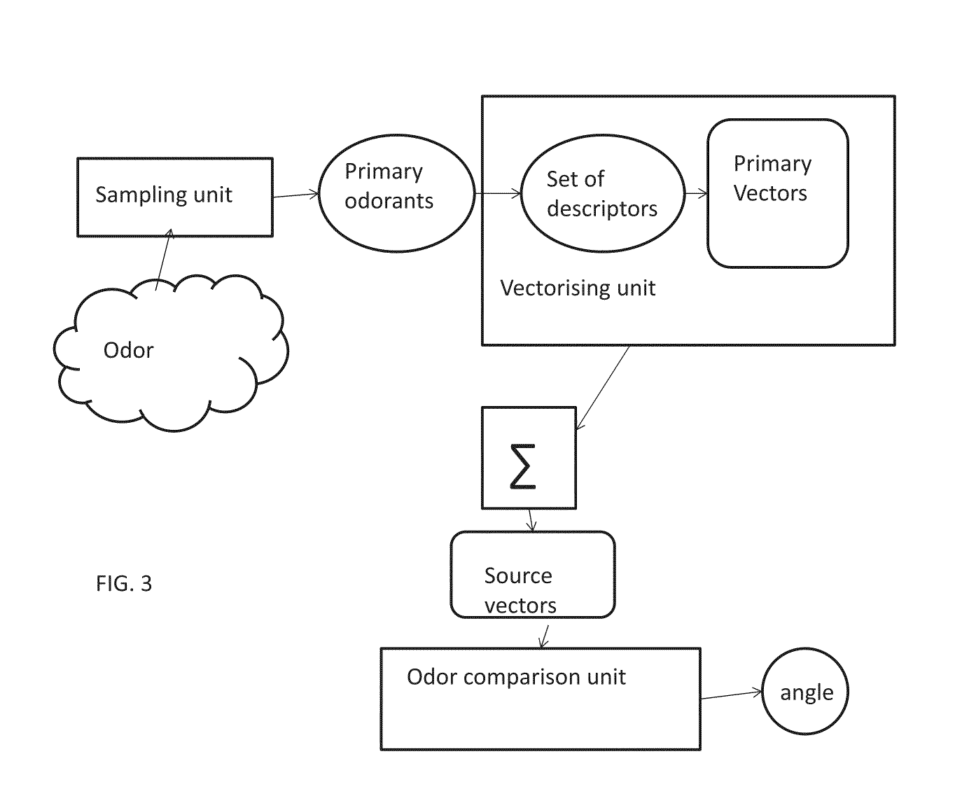

Reference is now made to FIG. 3, which is a simplified schematic diagram illustrating a detector which can detect primary odorants, based on a sampling device such as for example a gas chromatography mass spectrometer (GCMS), or an electronic nose for detecting and comparing odors according to embodiments of the present invention.

A sampling unit 30 samples odor sources and detects the primary odorants 32 therein. A vectorising unit 34 converts each detected primary odorant into a primary vector based on the set of descriptors 36 described above, so that each sampled odor is now a series of vectors, one for each primary odorant, and each vector has a numeric entry for each one of the set of descriptors.

A summation unit 38 builds a source vector for each detected odor source by summing the respective primary vectors, and normalizing the result as necessary. The result is a vector again having a numerical entry for each one of the set of descriptors, but in this case the numerical entry is the normalized sum of the corresponding entry for each one of the separate primary vectors.

An odor comparison unit 40 compares two detected odor sources by determining the angle between the respective source vectors. As explained in reference to FIG. 2, the dot product is obtained from the source vectors to be compared. The dot product may be normalized and then an inverse cosine operation may be used to recover an angle.

Now considering the embodiments in greater detail, as referred to in the background, the science of odors was connected to the ability to differentiate between one smell and another, and the present embodiments develop a computational framework and algorithm that looks at the molecular structure of two odors, and predicts their ensuing perceptual similarity. The algorithm may work for odors that are each composed of a mixture containing tens of different molecules, much like natural smells. The algorithms of the present embodiments are particularly useful in the case of mixtures and treat the odor-mixture as a single value, rather than a bunch of values reflecting each of its individual components. This is consistent with the growing view of how the mammalian brain treats odors: synthesizing a singular odor percept rather than analytically extracting individual odorant features from the odor-mixture. Thus the performance of an algorithm according to the present embodiments may contribute to the practice of the science of odor in general including the understanding of brain mechanisms of smell.

### Selecting Components for Odorant-Mixtures

Odorants can generally be described by a large number of perceptual or structural descriptors. Dravnieks' atlas of odor character profiles includes 138 mono-molecules, each described by 146 verbal descriptors of perception. This is an example of what we refer to herein as the ‘perceptual odor space’. Odorants can also be described by a large set of structural and physicochemical descriptors. We selected 1358 odorants commonly used in olfaction research, and obtained 1433 such descriptors using the Dragon software v. 5.4, of Talete s.r.l, Milan, Italy referred to above. It is noted that Dragon actually provides 1664 descriptors, but 231 descriptors are without values for the molecules being modelled.

Since the different descriptors measure properties on differing scales we normalize the Dragon data so that the values of each descriptor range between 0 and 1. That is, for each descriptor d we have a set of 1358 values ld, barring missing values. Each value v in the list ld is normalized to the value vn by the equation

$$\frac{v - min ( ld )}{max ( d ) - min ( ld )} \qquad ( Equation 1 )$$

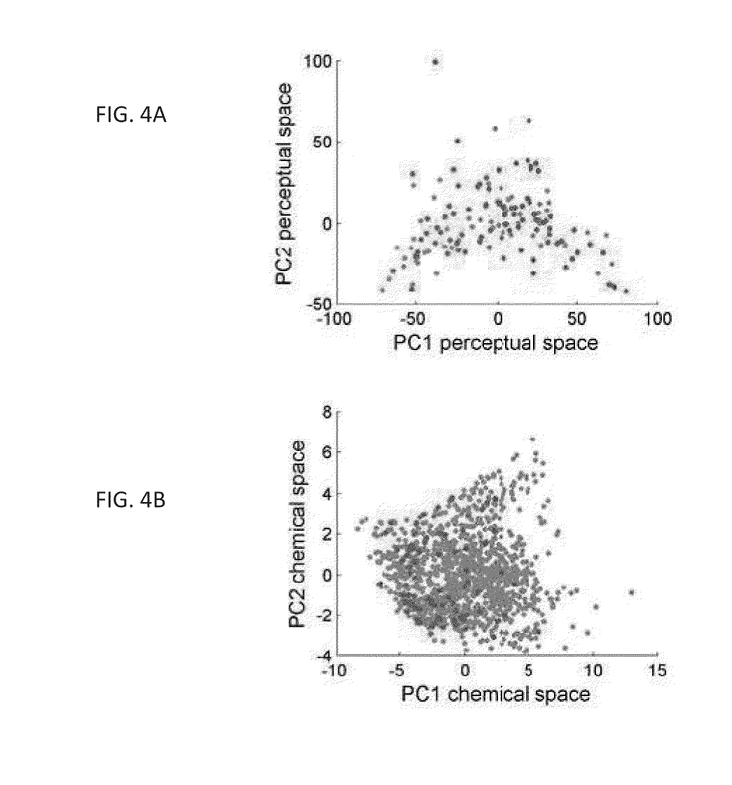

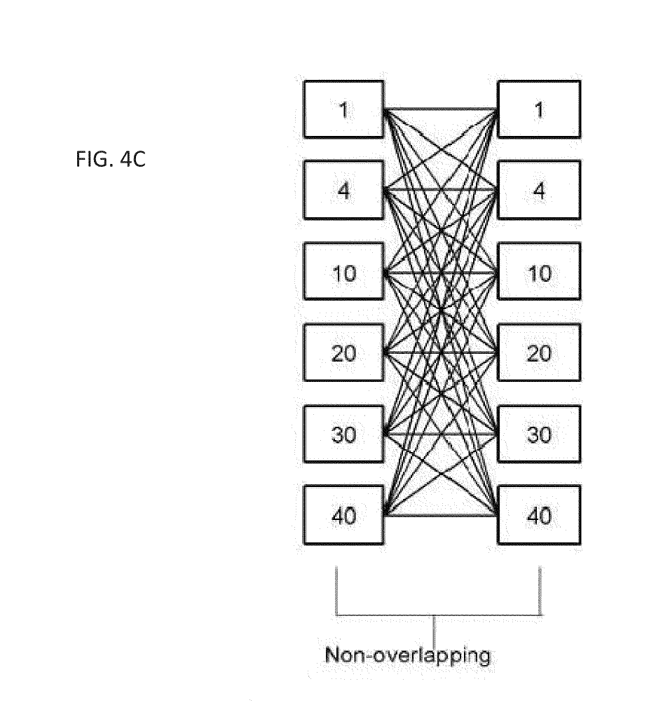

Reference is now made to FIGS. 4A, 4B and 4C which are graphs illustrating odorant selection and comparison. The odorants used are plotted in red, and presented in FIG. 4A within perceptual space. In FIG. 4A, 138 odorants commonly used in olfaction research are projected onto a two-dimensional space of PC1 (30.8% of the variance) and PC2 (12% of the variance) of perception. In FIG. 4B, the odorants are shown in physicochemical space: 1358 odorants commonly modelled in olfaction research are projected onto a two-dimensional space made of PC1 (37.7% of the variance) and PC2 (12.5% of the variance) of structure. FIG. 4C shows a schematic reflecting mixture comparisons in Dataset #1, see table below. Each mixture was compared to all other mixtures with zero overlap in component identity, and to itself. Note that this schematic reflects one quarter of the data, as we had eight versions of each mixture size.

The normalized data referred to herein is made up of the odorants in the physicochemical odor space of FIG. 4B and table S1 contains the odorants modelled and their descriptor values. To form odorant-mixtures, 86 mono-molecular odorants that were well-distributed in both perceptual (FIG. 4A) and physicochemical (FIG. 4B) stimulus space were used, as detailed in Dataset #1, hereinbelow. Each odorant was then diluted separately to a point of about equal perceived intensity as estimated by an independent group of 24 subjects, and various odorant mixtures containing different numbers of such equal-intensity odorant components were prepared. To prevent inadvertent formation of novel compounds, odorant mixtures were not mixed in the liquid phase, but rather each component was dripped onto a common absorbing pad in a sniff-jar, such that their vapors alone mixed in the jar headspace. The integrity of the present method was later verified using gas-chromatography mass-spectrometry (GCMS), as detailed in the section ‘methods’ hereinbelow. The present inventors prepared several different versions for each mixture size containing 1, 4, 10, 15, 20, 30, 40 or 43 components, such that half of the versions were well-spread in perceptual space, and half of the versions were well-spread in physicochemical space.

The present inventors then conducted pairwise similarity tests, using a visual analogue scale (VAS) as discussed in greater detail in the Methods section hereinbelow, of 191 mixture pairs, with 48 subjects of whom 24 were women, using an average of 14 subjects per comparison. Each target mixture (1, 4, 10, 15, 20, 30, 40 or 43 components) was compared to all other mixtures (1, 4, 10, 15, 20, 30, 40 or 43 components), and as a control, to itself. Other than comparisons of a mixture to itself (44 comparisons), all comparisons were non-overlapping (147 comparisons), i.e. each pair of mixtures under comparison shared no components in common (FIG. 4C). Table S2 contains all the similarity estimates for the three datasets used in this study.

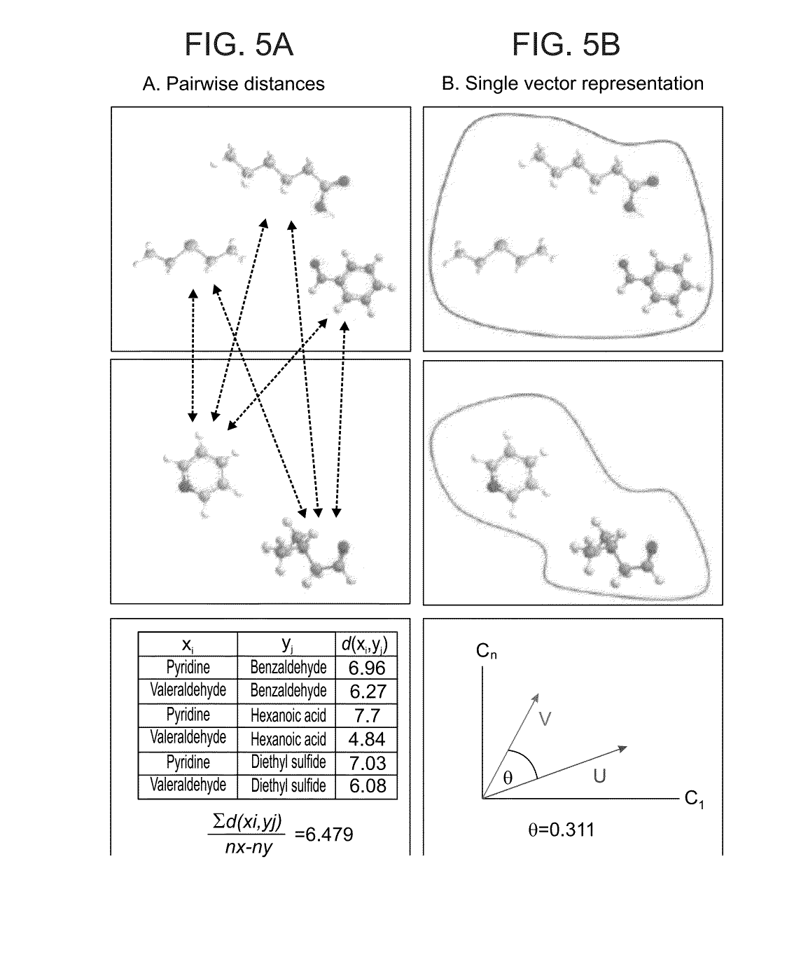

Reference is now made to FIG. 5 which is a schematic diagram showing modelling of odorant mixtures as singular objects rather than component amalgamations. The top panels represent one mixture (Y) made of 3 mono-molecular components and the bottom panels represent a different mixture (X) made of 2 mono-molecular components. The distance between X and Y can be calculated as (A) The mean of all pairwise distances between all the components of X and Y. (B) Alternatively, one can represent both X and Y as single vectors reflecting the sum of their components, and define the distance between them as the angle between these two vectors within a physicochemical space of n dimensions.

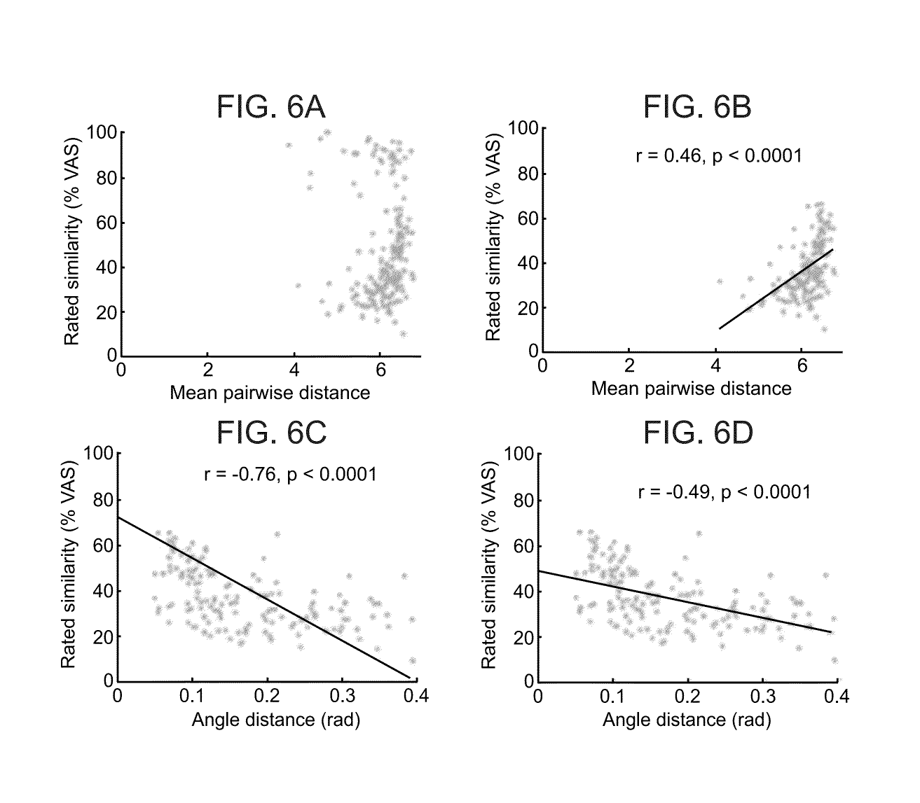

Reference is now made to FIGS. 6A to 6D, which are a series of graphs illustrating performance of the pairwise distance and angle distance models. Each dot reflects a comparison between two odorant mixtures. (A) The pairwise distance model was not predictive of mixture similarity. (B) Removing comparisons of a mixture to itself, the pairwise distance model implies a non-logical point from which increases in structural similarity drive decreases in perceived similarity. (C) The angle distance model provides a strong prediction of perceived similarity. (D) The angle distance model continues to provide logical results after removing comparisons of mixtures to themselves.

### The Pairwise Distance Model for Odorant-Mixture Similarity

One simple model for predicting the perceptual difference between mixtures is to measure all pairwise Euclidean physicochemical distances between all individual mixture components, and then average them. This approach treats each mixture component individually, as shown in FIG. 5A. To test this model, we obtain the 1433 physicochemical descriptors for each of the 86 mono-molecular components we used. We find that the mean pairwise Euclidean distance over all the descriptors of all mono-molecular components comprising any two mixtures is a poor predictor of perceptual similarity between the two mixtures. The relationship between pairwise-distance and perceived similarity does not fit any simple model, linear or other, as clear from FIG. 6A. Moreover, the distribution of this relationship is clearly skewed by the similarity ratings given to the comparisons of a mixture to itself, yet eliminating these comparisons reveals a significant correlation in the opposite direction (r=0.46, p<0.0001) as shown in FIG. 6B. In other words, the pairwise distance model implies that odor-mixtures identical in structure will be the furthest apart in perceptual similarity. Given this clear failing-point of the model, we investigate an alternative model.

### The Angle Distance Model for Odorant-Mixture Similarity

An alternative model is to consider the mixture as a whole rather than a set of constituents, as in FIG. 5B. To test such a model, we use the same 1433 physicochemical descriptors for each mono-molecular mixture component, but this time we create a single vector representing the whole mixture by summing the vectors of its components. To eliminate the effect of the number of components in a mixture on the size of the mixture vector, we divide the mixture vector by its norm. Thus, each mixture is now represented by a vector made of 1433 descriptors. We then define the distance between the vector of mixture U and the vector of mixture V, as the angle between the two vectors, given by:

$$θ ( \vec{U} \vec{V} ) = arccos ( \frac{\vec{U} . \vec{V}}{\vert \vec{U} \vert \vert \vec{V} \vert} ) \qquad ( Equation 2 )$$

where U·V is the dot product between the vectors, and |U|,|V| are the norms of the vectors. We find that the angle distance as defined by equation 2 is predictive of perceived mixture similarity (r=−0.76, p<0.0001) (FIG. 3C). Omitting comparisons of mixtures to themselves results in a correlation of r=−0.49, p<0.0001 (FIG. 3D). Unlike the pairwise distance model, this model does not predict that physically identical mixtures would in fact smell dissimilar. In the following some optimizations are provided of the angle distance model.

### Optimizing the Angle Distance Model

In order to optimize the model, we first set out to collect an independent dataset (Dataset #2). To address the possibility that the performance of our model is somehow influenced by the nature of our mixtures, whose components were selected to span olfactory space, the components for Dataset #2 mixtures are selected randomly. We randomly select 43 molecules out of the 86 equated-intensity molecules, and make 13 mixtures of 4-10 randomly selected components. Thus, unlike in Dataset #1, here there was some overlap in components across mixtures, rather more like odors in the real world. Twenty-four subjects, including 13 women, conducted pairwise similarity tests of all 91 possible pairs plus 4 comparisons of identical mixtures for a total of 95 comparisons, and each such comparison was repeated twice. Subjects conducted the similarity tests within four sessions on four consecutive days (−48 comparisons per day). Comparisons were counter-balanced for order.

### Model Optimization: Selecting Chemical Descriptors Through Simulation

The inventors extract the most relevant chemical descriptors for predicting perceptual similarity using the angle distance model. In order to do so, they compare the quality of predictions based on different combinations of descriptors. However, because the data includes 1433 different descriptors, it is impossible to compare all possible selections of descriptors in order to pick the best performing selection (2¹⁴³³ possibilities). With this in mind, we first set out to model the total number of descriptors our model may rely on.

### Step 1: Selecting the Number of Descriptors

The first step in the optimizing method is to decide on the number of features (descriptors) to look for. To do this we use a random half of Dataset #2 as a training-set (47 comparisons) and run a simulation.

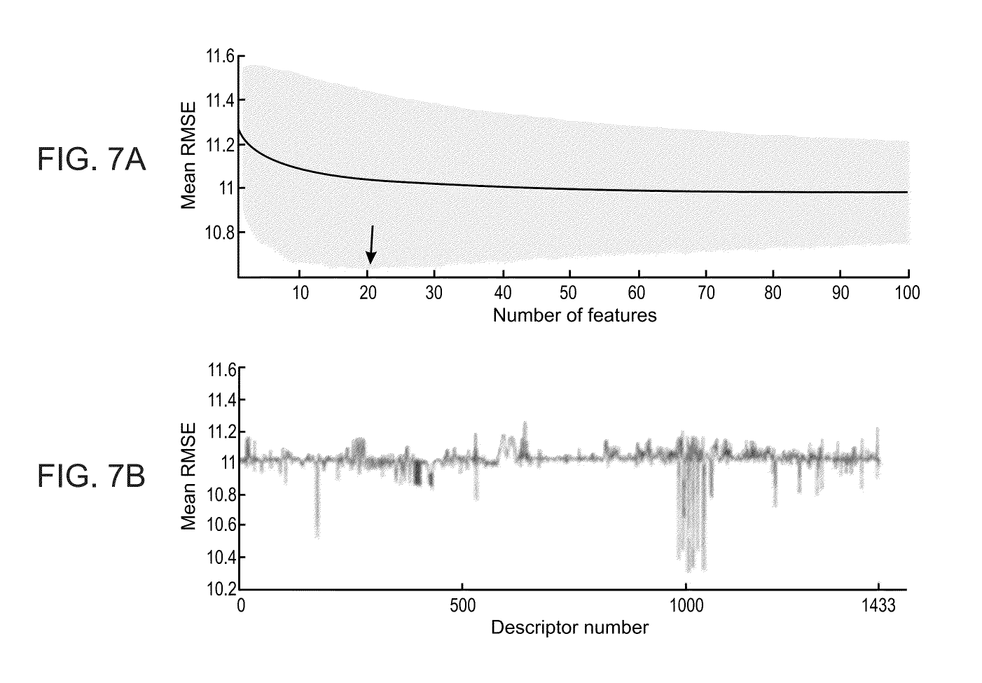

Reference is now made to FIGS. 7A and 7B, which are graphs illustrating optimizing the angle distance model.

FIG. 7A shows mean RMSE for varying numbers of descriptors, that is features. Plotted in grey are the standard error values for each number of features. The lowest value was obtained at about 20. FIG. 7B shows change in the mean RMSE for the individual descriptor. For each of the 1433 descriptors, the mean RMSE was calculated between the similarity ratings of mixture pairs and the angle distance model based on 2,000 selections of 25 random descriptors, one of which is the fixed descriptor in question. A score was given to each descriptor based on this mean RMSE for the next step.

In the simulation we run through each number of features from 1 to 100. For each number of features n we select 20,000 random samples of descriptors of size n and calculate the root mean square error (RMSE) for the prediction on the training set comparisons based on these descriptors. For each n we then calculate the mean RMSE and the standard deviation and plot the result, as shown in FIG. 7A. At n=20 the value of the mean RMSE minus the standard deviation is the Lowest. In FIG. 7A, the trend continues to increase for n>100. This indicates that at around 20 descriptors, we should expect the selections that would produce the lowest RMSE. Since our feature selection method includes the possibility of selecting a feature twice, we searched for slightly larger size sets of features so that the duplicates could be removed and at the end of the process we would have about 20 descriptors.

### Step 2: Evaluating Individual Descriptors

Although we may compare the performance of a selection of descriptors, we want to estimate the relevance of individual descriptors. If we select 25 descriptors at random out of the 1433 and base a predictive model on them, we are likely to obtain a prediction that correlates to an RMSE of about 11, as shown in FIG. 7A. However in order to optimize our model we want to distinguish those descriptors which give rise to more accurate predictions from those that do not. In order to evaluate a descriptor d in terms of how much it contributes to accurate predictions we run a simulation for each descriptor. In the simulation for descriptor d we test predictive performance of a large number of randomly selected sets of descriptors to which we add descriptor d. We use 2000 random selections of 25 descriptors together with d and test their predictive performance on the same training and testing set as before. For each selection we calculate the RMSE, and then calculate the mean RMSE across the 2000 selections. This mean is the number assigned to descriptor d (FIG. 7B), giving us an indication of how relevant the descriptor d is to making similarity predictions: the lower the mean RMSE, the more relevant d is. FIG. 7B is a plot of these averages calculated for each one of the 1433 descriptors. As apparent in the figure, for most descriptors the average performance for random selections that include them is about the same. However, some descriptors stand out.

### Step 3: Searching for the Best Selection of Descriptors

The next step in the descriptor selection process is a second simulation where we select 4000 samples of 25 descriptor sets based on the performance of the individual descriptors in the second step of the selection process. We give each of our descriptors a non-negative score based on its mean RMSE calculated in the first part of the process. The score is calculated as

score=max(0,−zscore(mean_RMSE)) (Equation 3)

so that only descriptors with an RMSE value lower than the average RMSE value (i.e. good-performing descriptors) are associated with a score greater than zero. Then we proceed to select random samples according to the scores just calculated. That is, in the third step of the process, those descriptors that performed better in the second step were more likely to be included in the (semi) random sample. Using this method we select 4000 samples of 25 descriptors and pick the ones that perform best, i.e. the selection that produces the lowest RMSE in the training set predictions. We remove repeated descriptors from our best performing selection of 25 descriptors and obtain a selection of 21 descriptors that perform even better (Table 1).

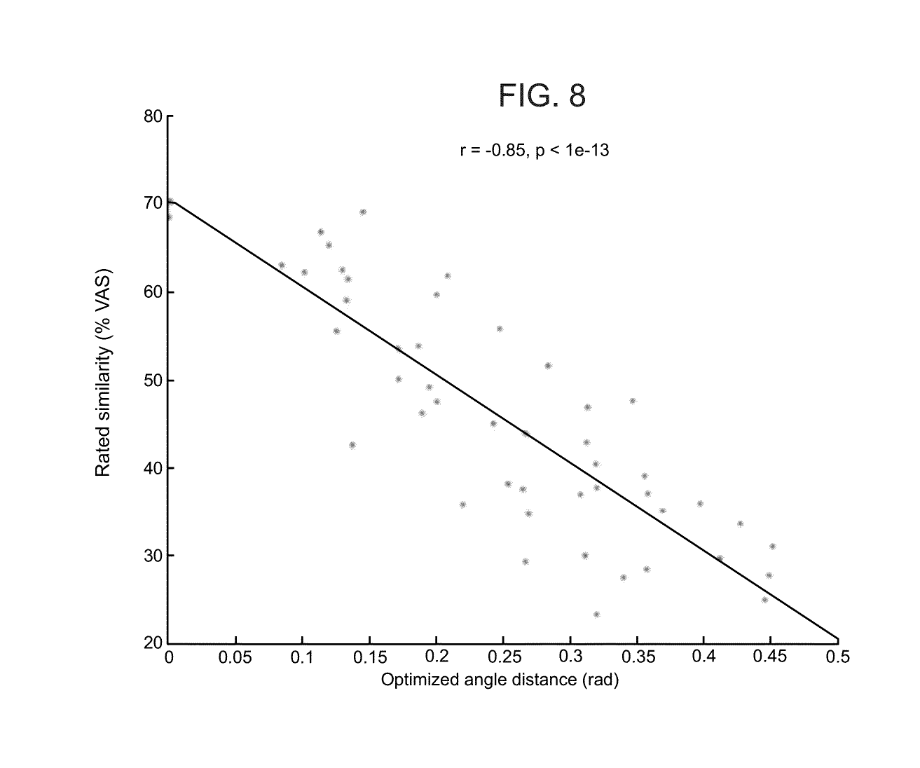

Reference is now made to FIG. 8, which is a graph illustrating performance of the optimized angle distance model. In FIG. 8, each dot represents a comparison between two mixtures. The optimized model may provide a strong prediction of mixture perceptual similarity from mixture structure alone. FIG. 8 illustrates the performance of the descriptors selected according to the above two-step training process being tested on the testing set. The resulting correlation between predicted odorant-mixture similarity and actual odorant-mixture similarity is RMSE=6.98, r=−0.85, p<0.001. Whereas the above random selection of descriptors may give rise to different descriptor subsets in recurring simulations, a deterministic selection of descriptors does not generate better results.

Further Optimizing by Selecting Chemical Descriptors Using Minimum Redundancy Maximum Relevance Feature Selection (mRMR)

The above-described selection of an optimized subset of descriptors involves random selections and may give rise to different descriptor subsets in recurring simulations. The present inventors thus set out to repeat the descriptor subset selection process using a different, deterministic method. To do so, a method was adopted that considers minimal mutual information between descriptors and the measure to be evaluated, i.e. rated similarity. The method uses a measure of mutual information to select the relevant features without redundancy, including information about the category of the observation to carry out the calculation. That is, in the present case the method uses information about the average rated similarity to select chemical descriptors relevant to it. The data for the program is a matrix of observations and a list of categories for each of the observations. In the present case the categories are the average rated similarities between mixtures and the data matrix describing the comparisons between the mixtures. The mutual information distance script mRMR_mid_d selects the best 25 descriptors based on the data matrix representing the comparisons in the training set. We test the performance of this selection on the testing set of comparisons in Dataset #2 as done for the previous method. The results give RMSE=11.5888 and r=−0.4908, p<0.005. This result was significantly poorer than that obtained with the optimized descriptor set. It should be noted that although the mRMR method uses information about the rated similarity to select descriptors it does not actually consider the measurement of prediction as we do in the simulation method.

### 2. Predicting an Olfactory White

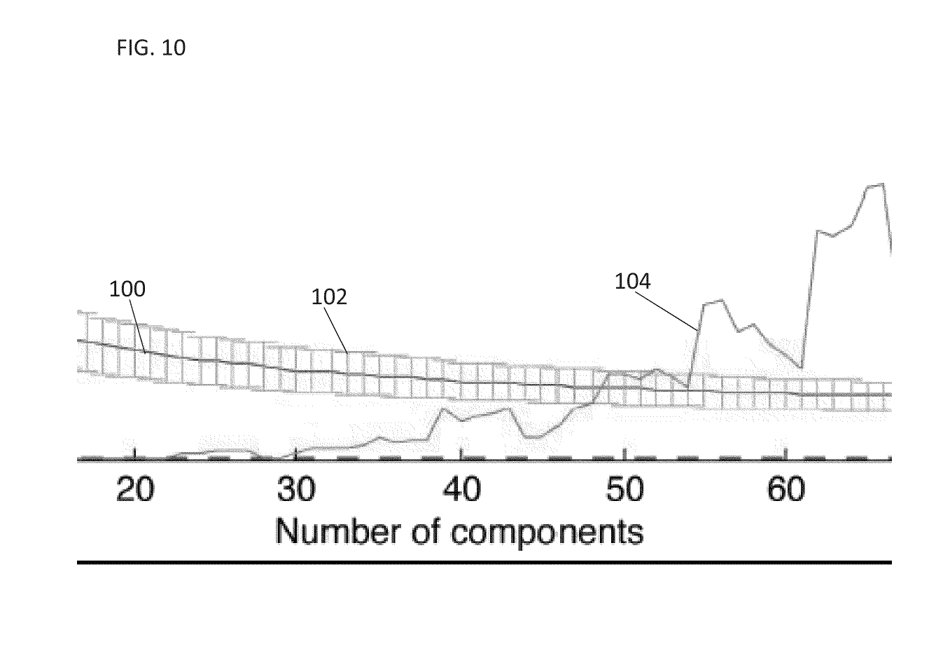

Reference is now made to FIG. 10, which is a graph predicting the presence of Olfactory White based on the number of components using the angle distance model. Line 100 shows the mean angle between a theoretical mixture made up of 679 monomolecular components, and other non-overlapping mixtures made of increasing numbers of components. In the experiment, 5000 randomly selected mixtures were made for each number of components on the horizontal axis from 2 to 80. Error bars 102 shows are STD. Line 104 is the p value for a t-test between consecutive mixtures, with a running average of five comparisons, and the test remains significant up to around 25 components but only rarely beyond 36 components.

As explained above, a prediction of the angle-distance model is the existence of a point, in terms of number of components, where all mixtures tend to smell similar, a point we may call olfactory white. According to our model, this point corresponds to the percept generated by a mixture having the mean values of each of the physicochemical features. To simulate this point, we calculate the coordinates of a mega-mixture containing 679 odorants, namely half of our available database. Next we calculate the predicted perceptual similarity between this mixture and increasingly large mixtures, each randomly selected 5000 times from the second half of the database, ensuring that the mixtures under comparison shared no components in common. We observed that the angle distance between the megamixture and mixtures of increasing size levelled off from as early as ≈30 components See FIG. 7A. To further estimate the point of levelling, we conduct t-tests on the predicted angle between the megamixture and consecutive odorant mixture sizes. The first point at which angles for consecutive mixture sizes are not significantly different is at 25 components, and from 36 components and more, consecutive mixtures are only rarely significantly different—See FIG. 7a. We conclude with a conservative estimate that predicted similarity begins to level off at 30±10 components. This suggests that any mixture of 30±10 components will be perceptually similar to any other non-overlapping mixture of 30±10 components, or phrased differently, a 30±10 point random sample is a sufficiently good estimator of the mean. These predictions, of course, assume that the components are well distributed in the physicochemical space, and are of equal perceived intensity.

### The Model Predicted Similarity in Separate Datasets

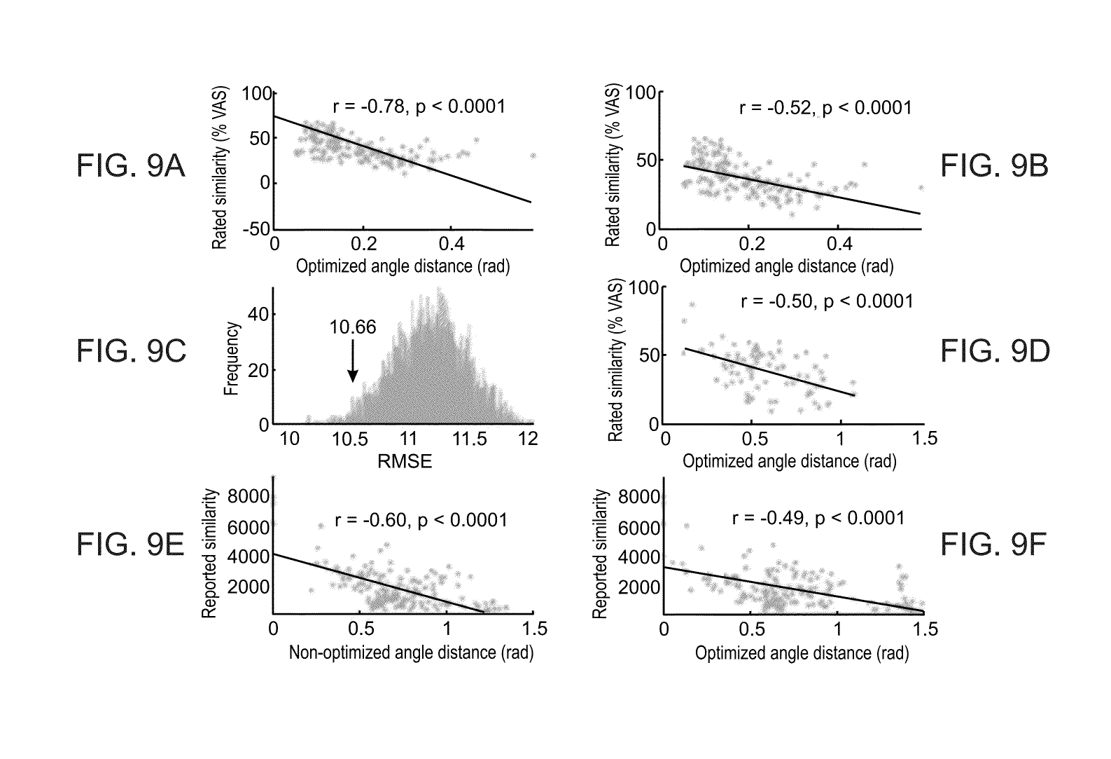

One might ask how well the present model performs under different conditions. Recall that so far the model has been optimized on Dataset #2 consisting of mixtures ranging in size from 4 to 10 components. Reference is now made to FIG. 9 which illustrates performance of the optimized angle distance model on independent data. FIG. 9A illustrates performance of the optimized model on complete Dataset #1. Each dot reflects a comparison between two mixtures. FIG. 9B shows the same as in FIG. 9A after omitting comparisons of mixtures to themselves. FIG. 9C is an RMSE histogram reflecting the performance of random selections of 21 descriptors. The optimized selection was at an RMSE of 10.66, which is better than 95.30% of the randomly selected sets. FIG. 9D shows performance of the optimized angle distance model on mono-molecules (Dataset #3). FIG. 9E illustrates performance of the angle distance model on mono-molecules tested 50 years ago independently by others. FIG. 9F illustrates performance of the optimized angle distance model on the data in FIG. 9E. Each dot reflects a comparison between two mono-molecules.

We now set out to test the performance of our model and selected descriptors on Dataset #1. This set not only includes larger mixtures but also includes 43 additional molecules not included in Experiment 2. Using Dataset #1 we obtain a correlation of r=−0.78, p<0.0001 for all comparisons (FIG. 9A), and r=−0.52, p<0.0001 for non-overlapping comparisons alone (FIG. 9B). To further get a sense of how well this selection of descriptors performs on the enlarged data, we compare its performance to that of 4000 randomly selected sets of 21 descriptors. We measure the performance in terms of RMSE on Dataset #1. The selected set of 21 descriptors predicts similarity with an RMSE of 10.66. Compared to randomly selected sets of descriptors, the optimized set performs better than 95.30% of the randomly selected sets (FIG. 6C).

Performance was tested using only the 147 comparisons between non-overlapping mixtures.

### The Model Predicts Similarity in Mono-Molecules

One may ask how a model that was optimized and tested in odorant-mixtures performs with mono-molecules. To obtain similarity ratings for mono-molecules we pool three experiments to form Dataset #3. The first experiment includes similarity ratings by 21 subjects, of whom 11 are female, between 14 pairs of mono-molecules; the second includes similarity ratings by 17 subjects, of whom 9 are female, between 20 pairs of mono-molecules, and the third includes 19 subjects, of whom 6 are female, rating 40 pairs of mono-molecules for similarity. In total, 49 mono-molecules are included in the present experiment. The pool of molecules is included in the original pool of 86 molecules in Experiment #1 and includes 42 of the 43 in the pool of Experiment #2. In total, 74 comparisons are conducted amongst the 49 molecules. Out of these comparisons, 65% (48 comparisons) include at least one molecule that was not used in Experiment #2. Each comparison is repeated twice.

We apply our selected set of descriptors to Dataset #3. As before, we measure the RMSE of the prediction made based on the descriptors we select. We obtain an RMSE of 13.825 and r=−0.5, p<0.0001 (FIG. 9D). In comparison, using all descriptors gives r=−0.39, p<0.0001. Thus, the set of descriptors optimized on Dataset #2 improves the predictive performance of the present model on Dataset #3. Notably, Dataset #3 consists of 7 additional molecules that were not included in Dataset #2 which was used to optimize the model. Moreover, as previously noted, 65% of these comparisons include at least one molecule that was not used in Experiment #2. This renders the test on Dataset #3 fairly unrelated to the set of molecules used to optimize the model.

The Model Predicts Similarity in Mono-Molecules Studied Independently

If the present model is to be helpful to researchers in the field, it must be applicable to data collected by others. Most published studies on olfactory mixtures look only at simple mixtures of 2 to 4 components, and moreover, most do not post their raw similarity matrices. The lack of posted raw data holds true for most studies of mono-molecular perceptual similarity as well, with one notable exception that we are aware of: Wright and Michels (1964) printed a large table containing the pairwise similarity ratings given by 84 subjects to a matrix of odorants that included 33 odorants not in our experiments or model building. We apply our model to their data. The angle-distance model, whether using the non-optimized or optimized descriptor set, yields a significant correlation between predicted and actual pairwise odorant similarity (non-optimized: r=−0.60, p<0.0001 (FIG. 9E); optimized: r=−0.49, p<0.0001 (FIG. 6F); difference between r values: z=−1.34, p=0.18). Thus, whereas Wright and Michels failed to predict perceptual similarity in their data, our model was a significant predictor of similarity in this data collected half a century ago. The statistically equal performance across the optimized and non-optimized descriptors when applied to this dataset may have resulted from several factors, including that the odorant selection criteria may have reflected the theory they were testing, that the molecules were not first diluted to equated intensity, and that these were indeed mono-molecules whereas our optimization was for the prediction of mixtures. However, the most likely explanation for this relates to their testing procedure: they compared similarity of all odorants to five anchor odorants. The five anchor odorants, by definition, are a skewed representation of olfactory space. Therefore, we take this as a reminder that researchers who set out to use the current model should consider both its optimized and non-optimized versions, especially in cases where the data may be skewed in olfactory space.

Descriptors that Predict Neural Activity were Poorer Predictors of Perceptual Similarity

Based on measures of neural activity and receptor responses, primarily in rodents, but also in humans, two independent studies obtained two alternative sets of optimal physicochemical odor descriptors. We set out to compare the performance of these sets of descriptors versus the current descriptors in predicting perceptual similarity. Application of the Haddad descriptor set (containing 32 descriptors) and the Saito descriptor set (containing 20 descriptors) to the testing set of Dataset #2 yielded RMSE=12.4049, r=−0.3608, p=0.01 and RMSE=11.2255, r=−0.5364, p<0.0001, respectively.

Although significant, these predictions are significantly weaker than those obtained with the optimized angle distance model (difference between r values, both z>3.16, both p<0.005).

In further work, parallel experimentation was carried out. The present computational model predicts the perceptual similarity of odorant mixtures and its nature implies that odorant mixtures form a single unified percept rather than a collection of components.

As explained above, as real-world odorants are almost never composed of a single molecule, it might be that important features of odorant perception are only apparent in mixtures. For that reason and in the hope of generalizing the models that exist for single molecule odorants, the present embodiments as discussed investigate the similarity of intensity equated odor mixtures. The present embodiments may provide a model that works consistently well under differing conditions such as the size of the mixtures and the selection of odorants in the sample pool.

The present inventors conducted three similarity experiments. The experiments vary in the composition of the odorants and in the size of the mixtures. The results from the three experiments (described below) are labeled datasets A, B and C. The first stage of the project is to pick the best performing model for predicting odorant similarity. We compare different models' performance on dataset A. Having found the angle distance model as discussed above to be the best performing model, we collect new data with greater accuracy in datasets B and C and used dataset B to optimize the present model and improve its performance. Finally, the optimized model is retested on datasets C and A.

### Experiment A

We obtain 86 monomolecular odorants that are well distributed in both perceptual and physicochemical stimulus space. We then dilute each of these odorants separately to a point of about equal perceived intensity as estimated by an independent group of 24 subjects, and prepare various odorant mixtures containing various numbers of such equal-intensity odorant components. To select the components of each mixture, we use an algorithm that automatically identifies combinations of molecules spread out in olfactory stimulus space. We prepare several different versions for each mixture size containing 1, 4, 10, 15, 20, 30, or 40/43 components, such that half of the versions are optimally spread in perceptual space, and half of the versions are optimally spread in physicochemical space. We conduct pairwise similarity tests, using a 9-point visual analogue scale; VAS of 191 mixture pairs, in 56 subjects and using an average of 14 subjects per comparison. Each target mixture (1, 4, 10, 15, 20, 30, or 40/43 components) was compared to all other mixtures (1, 4, 10, 15, 20, 30, or 40/43 components), and as a control, to itself. Other than comparisons of a mixture to itself, all comparisons were non-overlapping, in other words, each pair of mixtures under comparison shared no components in common. In total, the Experiment's dataset included 191 comparisons, 147 of which were non-overlapping and 44 of which were comparisons of a mixture to itself.

### Experiment B

The preparation of the mixtures follows the same method as in experiment A but we increase the accuracy of the data in two ways. First, we increase the number of participants to 24 subjects per comparison. Second, to negate the possibility of formation of new chemical entities due to interactions between the selected components, all mixtures are analyzed in gas chromatography mass spectrometry. The mixtures are analyzed both before and after heating (60° for 3 hours), as to enhance any chemical interactions that should have taken place only after a certain amount of time. Two mixtures out of the 14 tested show a retention time that does not match any of their components and are thus replaced. The replacement mixtures are similar to the replaced mixtures, except for one component whose retention time was missing in the analysis. The replacement mixtures were tested again in a similar manner.

We conduct pairwise similarity tests of all 91 possible pairs plus 4 comparisons of identical mixtures for a total of 95 comparisons. The tests are conducted using a continuous visual analogue scale (VAS) in 24 subjects. Each such comparison is repeated twice. Since the overall number of mixtures is rather small, we make two different jars for each mixture, which are labeled differently. In addition, four similarity tests are conducted between two identical mixtures. For these self-comparisons we select the two versions of four-component mixtures and the two versions of ten-component mixtures. Subjects conducted the similarity tests within four sessions on four consecutive days, in which 48 comparisons were made on each of two days and 47 on each of the two other days. Comparisons were counter-balanced for order. In total 43 molecules out of the original pool in experiment A were used in this experiment.

### Experiment C

This similarity experiment of mono-molecules consists of three different sets of experiments. The first experiment included similarity ratings by 21 subjects, including 11 female, between 14 pairs of molecules; the second included similarity ratings by 17 subjects, 9 being female, between 20 pairs of molecules, and the third included 19 subjects, 6 being female, rating 40 pairs of molecules for similarity. In total, 49 mono-molecules were included in this experiment. The pool of molecules is included in the original pool of 86 molecules in experiment A and includes 42 of the 43 in the pool of experiment B, and another 7 which are not included in experiment B. The procedure for preparing the mixtures and rating similarities followed the higher accuracy design of experiment B except that since the odorants are single molecules there was no need to test them with the gas spectrometer. In total, 74 comparisons were conducted amongst the 49 molecules. Out of these comparisons 65% (48 comparisons) included at least one molecule which was not used in experiment B. Each comparison was repeated twice under different labels.

### Odorant Mixture Similarity Model

The process which leads us to select the best performing modeling method is as described hereinabove and is based on the dataset of experiment A. We obtained a set of 1433 physicochemical descriptors of the molecules' structure. The values of each descriptor were normalized between zero and one to eliminate a scaling effect. An initial step in modeling similarity of two odorant mixtures is to find the best representation of the physicochemical data which describes it, that is the collection of chemical properties of each of the components which make up the mixture. There are two basic approaches to representing the data: the first approach, the ‘pairwise distance model’, treats a mixture as a collection of components and calculates its distance to other mixtures based on pairwise Euclidean distances between all molecules in both mixtures. The second approach is to represent a mixture by integrating and synthesizing the descriptors of its components into a single unified entity.

### Pairwise Distance Model

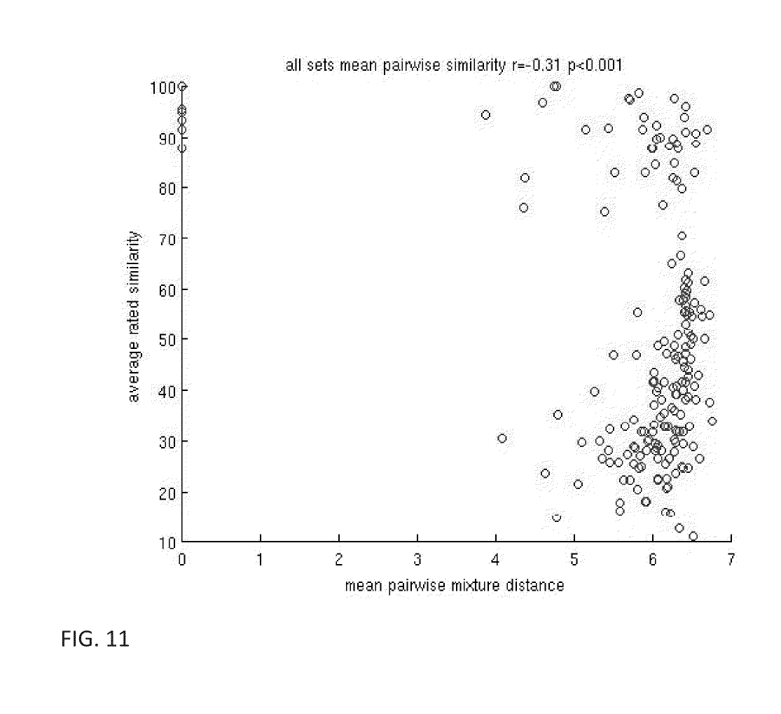

Referring now to FIG. 11, the mean pairwise distances are plotted against average rated similarity (experiment A).

The simple pairwise distance model treats each mixture component individually. To get a measure of the distance between two mixtures according to this model, all pairwise Euclidean distances between the components in one mixture and the components in the other mixture are averaged, where the vectors are the physicochemical properties obtained for each component. This approach treats each mixture component individually. We found that the mean pairwise Euclidean distance was a statistically significant yet weak predictor of perceptual similarity (r=−0.3, p<0.001, FIG. 11. One can claim that the correlation is mainly held by comparisons between identical single molecule mixtures, which are rated highly by subjects and are given a distance of zero according to the model. After eliminating these data points, the model provides no correlation to ratings (r=−0.04, p=−0.54) (FIG. 11). In other words, the prediction of this model would imply that as the mean of pairwise Euclidean distances increases, the mixtures are more similar to each other.

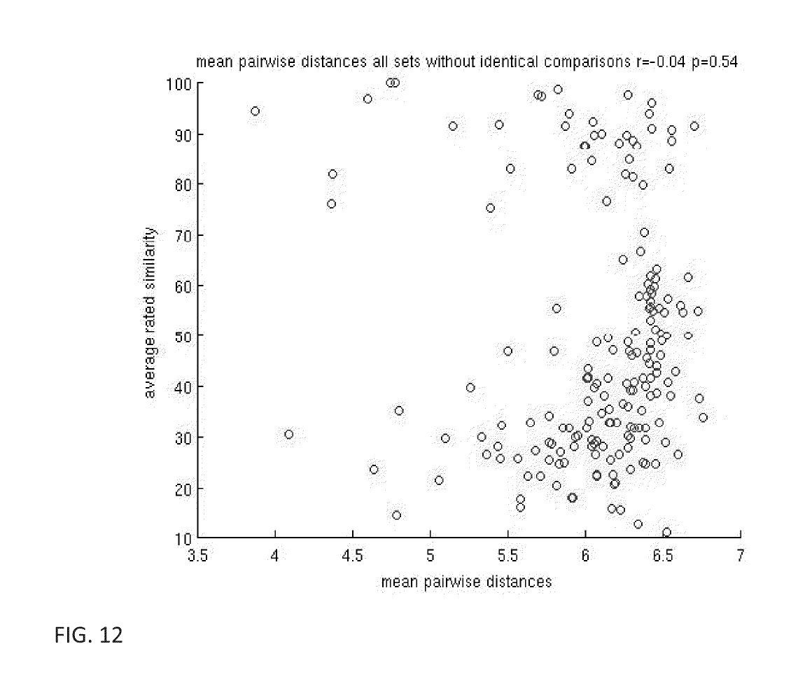

Reference is now made to FIG. 12, which shows the same comparison as in FIG. 11 but with the identical comparisons removed.

### Component Sum—Dot Product Model

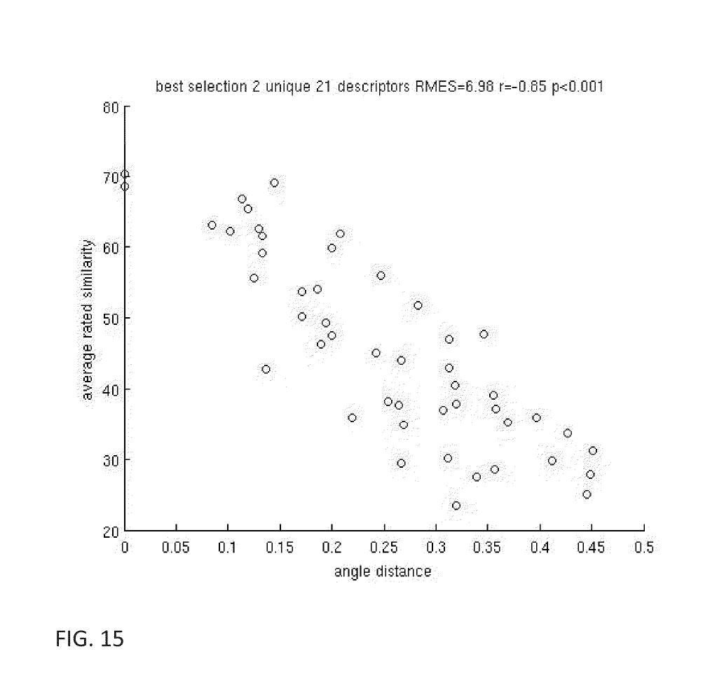

An alternative model is to consider the mixture as a whole rather than a set of its components. We used the same set of descriptors for each molecular component, and represented a mixture as the sum of its components' vectors. Thus, each mixture was now represented by a vector of 1433 values, and the values lost their original meaning as they were summed over a varying number of vectors. The distance between two mixtures according to this model is defined as the dot product of their vectors. Graphs of average rating against angle distance are shown in FIGS. 15, 16 and 18.

### Angle Distance Model

The component sum model does not take into account the number of components included in each of the two mixtures. Thus, a mixture which includes a large number of components will be represented by a vector with relatively large values. To eliminate this bias from the model we normalized each mixture vector by its norm. This normalized dot product is in fact the cosine of the angle between the two mixture vectors. Thus a modification of the dot product model leads to an angle distance model, where we defined the distance between two mixtures vectors as the angle between their vectors.

Recall that the angle between vectors u and v is given by

$$cos α = \frac{\vec{u} . \vec{v}}{\vert \vec{u} \vert \vert \vec{v} \vert}$$

### Selecting Chemical Descriptors Through Simulation

Having settled on an angle-distance model for predicting rated similarity we proceeded to optimize this model for best performance. We used a higher accuracy data set obtained in experiment B and consisting of 95 comparisons. We used a method designed to extract the most relevant chemical descriptors for predicting perceptual similarity using the angle distance model. In order to do so, we need to compare the quality of predictions based on different combinations of descriptors. However, since the data includes 1433 different descriptors, it would be impossible to compare all possible selections of descriptors in order to pick the best performing selection.

### Step 1: Selecting the Number of Descriptors.

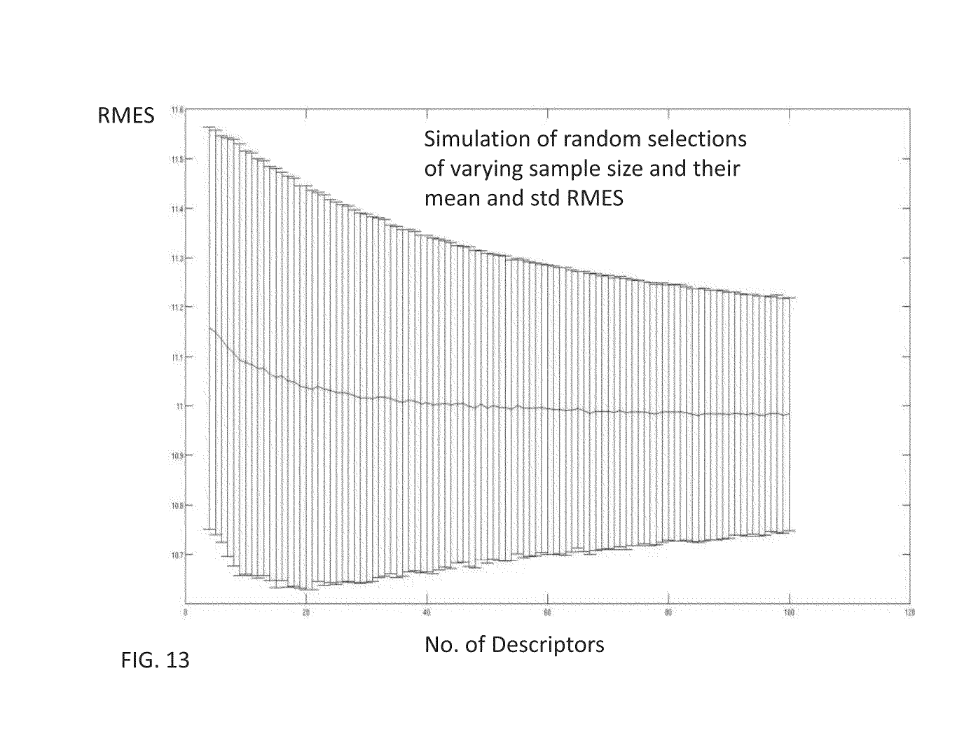

The first stage of our optimizing method is to decide on the number of features we are going to look for. To do this we used a random half of the data as a training set of 47 comparisons, and ran a simulation on it. In the simulation the present inventors ran through each number of features from 1 to 1000. For each number of features n the present inventors selected 20000 random samples of size n and calculated the root mean square error (RMSE) for the prediction on the training set comparisons set based on these descriptors. For each n the present inventors then calculated the mean of the RMSE and the standard deviation and plotted the result, and the results are shown in FIG. 13, to which reference is now made.

FIG. 13 illustrates that the minimum point of mean minus standard deviation is at n=20.

One can see that at n=20 the value of the mean of the RMES minus the standard deviation is the lowest (the graph continues to increase for n>100). This tells us that at around 20 descriptors, we can expect the selections which will produce the lowest RMES. Since the present feature selection method includes the possibility of selecting a feature twice we searched for slightly larger size sets of features so that at the end of the process we will end up with close to 20 descriptors.

### Step 2: Evaluating Individual Descriptors

Although we can compare the performance of a selection of descriptors we would like to know how relevant individual descriptors are.

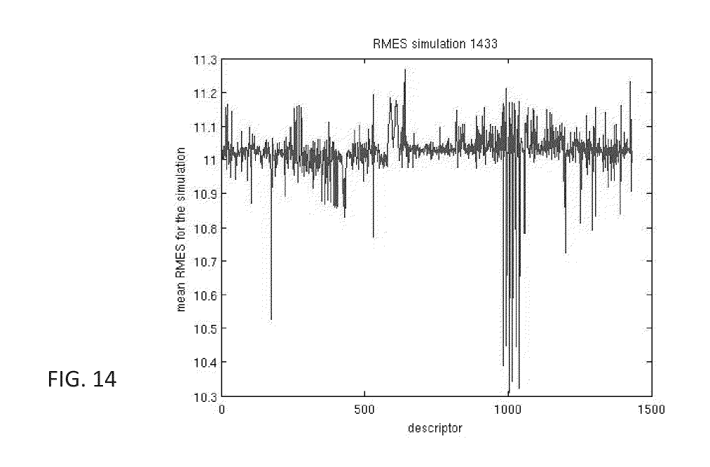

In this connection, reference is now made to FIG. 14. If we select 25 descriptors at random out of the 1433 and base our predictive model on them we are likely to obtain a prediction which correlates to an RMSE of about 11. In order to evaluate the relevancy of a certain descriptor d we considered the quality of predictions made by randomly selected sets of 25 descriptors together with d. We used the same training set and testing set from before. We then evaluated the performance of the model with these descriptors in predicting the similarity of the comparisons in the training set. We did this by selecting 2000 random selections of 25 descriptors amongst descriptors other than d, and for each one of them combined them with d and calculated the RMSE to the training predictions obtained by our model based on these descriptors. We averaged the RMSE obtained for each of the 2000 random selections to obtain an average correlation for random samples containing d. This gives us an indication of how relevant the descriptor d is to making predictions. FIG. 14 is a plot of these averages calculated for each one of the 1433 descriptors. As apparent in the figure, for most descriptors the average performance for random selections which include them is about the same. However, some descriptors stand out.

### Step 3: Searching for the Best Selection

The next stage in our descriptor selection process was a second simulation where we selected 4000 samples of 25 descriptor sets based in part on the performance of the individual descriptors in the first stage of the selection process. We gave each of our descriptors a positive score based on its mean RMSE calculated in the first part of the process. The score was calculated as

score=max(0,−meanRMESzScore),

so that those descriptors with a low (i.e. good) RMSE value were associated with a high score. Then we proceeded to select random samples according to the scores we just calculated. That is, in the second stage of the process those descriptors which performed better in the first stage were more likely to be included in the semi-random sample. Using this method we selected 4000 samples of 25 descriptors and picked the ones which performed best, i.e. the selection which produced the lowest RMSE in the training set predictions. We removed repeated descriptors from our best performing selection of 25 descriptors and obtained a selection of 21 descriptors which performed even better [see table ‘descriptors’ for a list of the descriptors]. The performance of the descriptors selected according to this two-stage training process was tested on the testing set and the results were RMSE=6.98 r=−0.85 p<0.001, as shown in FIG. 15.
FIG. 15 shows results using one set of descriptors, that were used to obtain the prediction.

### Testing Our Model on Other Data Sets

### 1) Larger Mixtures (Dataset A)

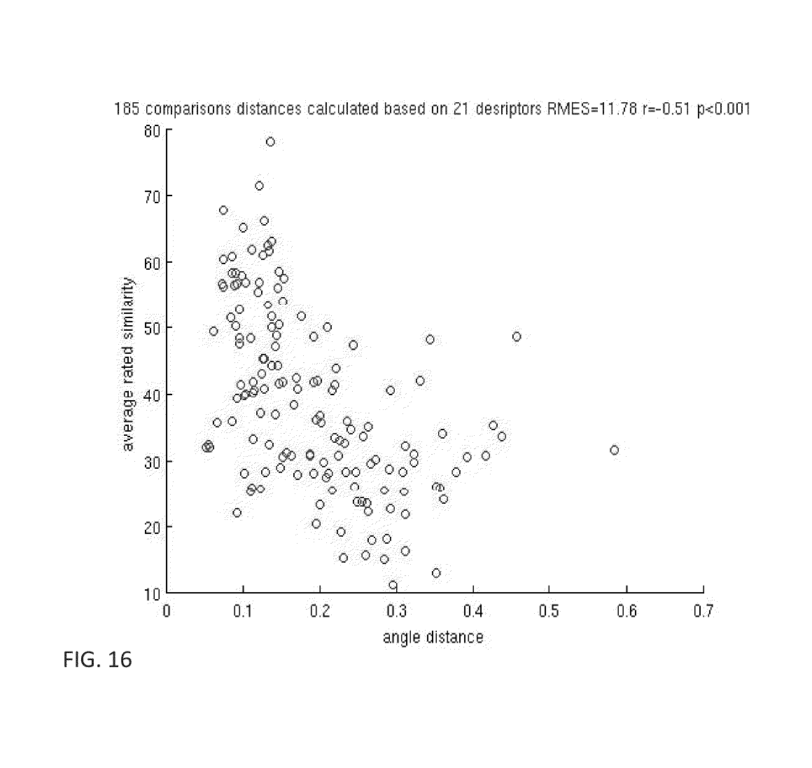

As discussed above, one might ask how well our model performs under different conditions. Recall that so far we have optimized our model on dataset B consisting of a pool of 43 molecules and mixtures ranging 4-10 components. To test this we retested the performance of our model and the descriptors we selected on dataset A. This set not only includes larger mixtures but also includes 43 additional molecules not included in experiment B. Using this set we obtained an RMSE of 11.7824 and a correlation of r=−0.51 p<0.001. See FIG. 16. FIG. 16 shows the angle distances are based on the 21 best descriptors selected based on the training set of the other set of data.

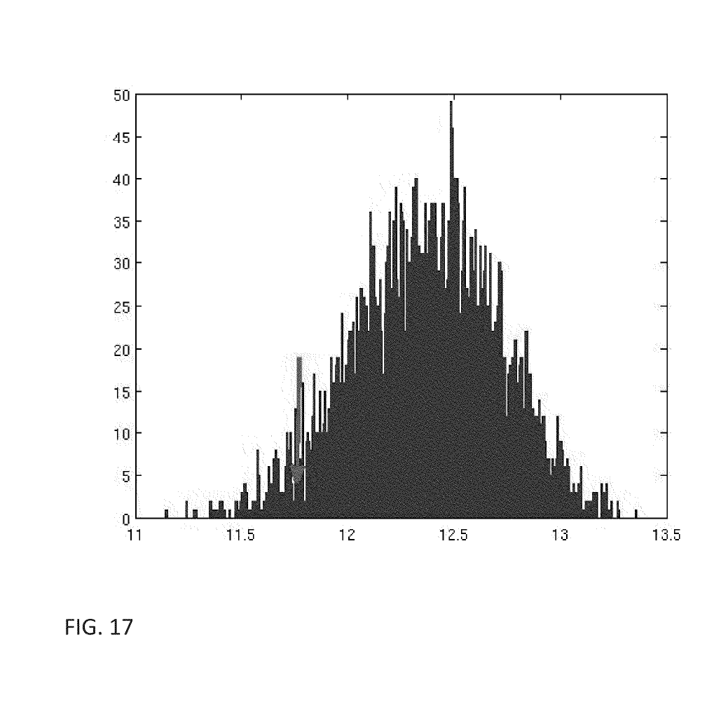

To get a sense of how well the present selection of descriptors performs on the data, we compared its performance to that of 4000 randomly selected sets of 21 descriptors. We measured the performance in terms of RMSE on dataset A and the set selected by training with an RMSE of 11.78 performed better than 95.04% of the randomly selected sets. The results are shown in the RMSE histogram of FIG. 17. The optimized selection was at 11.78 which is better than 95.04% of the randomly selected sets.

### 2) Mono-Molecules (Dataset C)

We applied our selected set of descriptors to dataset C. Recall that it consists of a collection of 74 comparisons between mono-molecules. The molecules were drawn from the same pool of molecules used for the previously discussed optimizing experiment. As before we measured the RMES of the prediction made based on the descriptors we selected. We obtained an RMSE of 13.825 and r=−0.49 p<0.001.

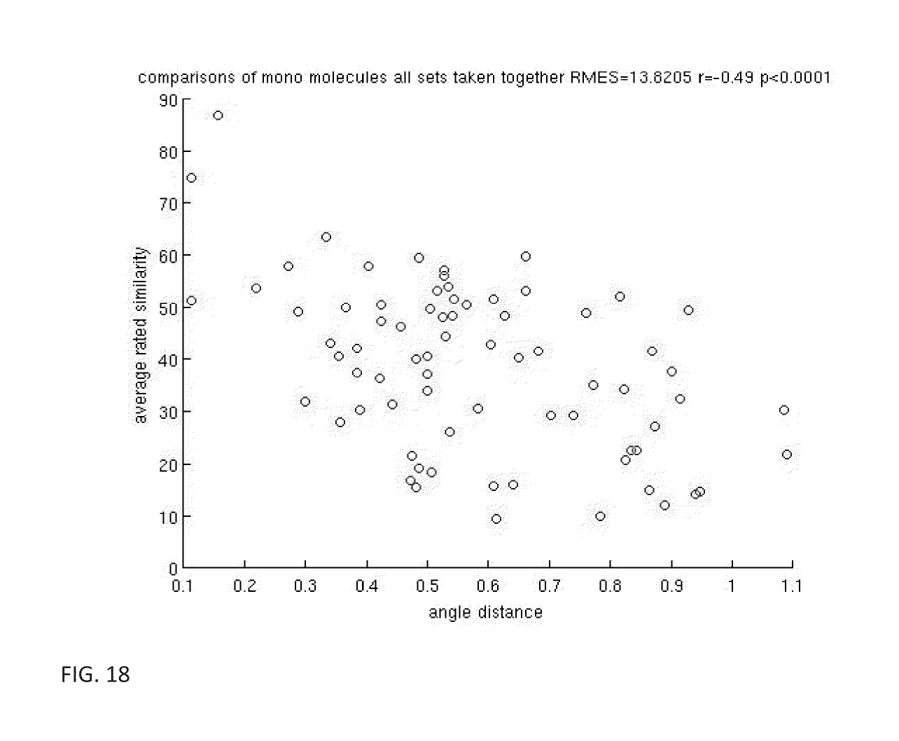

FIG. 18 illustrates the selected 21 descriptors tested on 74 comparisons of mono-molecules.

It should be pointed out that this dataset C consists of 7 additional molecules which were not included in dataset B which was used to optimize the model. Furthermore, as we mentioned above, out of these comparisons, 65% (48 comparisons) included at least one molecule which was not used in experiment B. This makes the test on dataset C fairly unrelated to the set of molecules used to optimize the model.

It should also be noted that as far as we know this is the first time that a model which can predict the rated similarity between single molecules was found.

Selecting Chemical Descriptors Using mRMR (Minimum Redundancy Maximum Relevance Feature Selection)

The present method uses a measure of mutual information to select the relevant features without redundancy. It uses information about the category of the observation to carry out the calculation. That is, in the present case the method uses information about the average rated similarity to select chemical descriptors relevant to it. The data for the program is a matrix of observations and a list of categories for each of the observations. In the present case the categories were the average rated similarities between mixtures and the data matrix described the comparisons between the mixtures. The way the data matrix represents the comparisons between the mixtures is as follows. The present model is an angle distance model between vectors representing mixtures, the angle between the vectors is calculated based on the inner product of the two vectors, and therefore the data matrix representing the comparisons between the mixtures contained the point-wise products of the vectors representing mixtures. So if the first comparison was between mixture A and mixture B represented by vectors V_a and V_b, the first row in the data matrix was the pointwise product of V_a and V_b.

The present model may use a mutual information distance to select the best 25 descriptors based on the data matrix representing the comparisons in the training set. The descriptors selected are as described above. The present inventors tested the performance of this selection on the testing set of comparisons in dataset B as for the other method. The results were RMSE=11.5888 and r=−0.4908 p<0.005.

It should be noted that although the mRMR method uses information about the rated similarity to select descriptors is does not actually consider the measurement of prediction as we do in the simulation method.

### Molecular Biology Implications

The present results show that a certain set of physicochemical properties of molecules are particularly relevant for predicting odorant similarity. Since the set of initial descriptors is highly redundant, the resulting subset of descriptors is not unique but it does perform far better than a random selection. It would be natural to consider the resulting subset and see if their relevance could be explained by molecular biology or suggest some hypothesis in molecular biology. Conversely, a hypothesis about a molecular biological process connected to olfaction can imply a set of relevant physicochemical descriptors. That hypothesis can be tested by testing the performance of the selected set of descriptors as predictors of odorant similarity in our model.

### DISCUSSION

In this disclosure the present inventors identify a model that allows predicting odorant-mixture perceptual similarity from odorant-mixture structure. The immediate impact of such a result may lie in the design of olfaction experiments probing both perception and neural activity, which can now be linked within a measurable predictive framework to the structure of odorant-mixtures. For example, one prediction of the model pertaining to mixtures that span olfactory space was that as the number of independent mono-molecular components in each of two mixtures increases, the two mixtures should gain in similarity, despite containing no components in common. In fact, the model predicted that at around 30 mono-molecular equally-spaced components, all mixtures should start smelling about the same We recently verified this prediction, which culminated in the odor Olfactory White.

### Why the Angle Distance Model

One may argue that there are countless potential paths to model the contribution of the various physicochemical descriptors to the perception of similarity, and therefore ask why an angle distance model was selected. Here the present inventors describe the evolution of the angle distance model over the course of the research effort: The simplest and most naive initial solution to the problem addressed was the pairwise distance model, and initial efforts centered on its optimization. The main weakness of the pairwise distance model is, as previously noted, its implication that the more common molecules two mixtures share, the more different they will smell. This is not a problem in the lab, where one can select non-overlapping mixtures (e.g., Dataset #1). In the real world, however, many different mixtures will typically share many common components (e.g., Dataset #2). The issue was initially tackled by adding a parameter that assigned a variable weight to the distance between components of one mixture that were close to components of the second mixture. A second parameter was added to define a threshold for being considered a close point. The added parameters were optimized but the performance of the model did not improve and inconsistencies remained.

In an attempt to further generalize the pairwise distance model the inventors then tried replacing the Euclidean distance that defines the pairwise distance with other typical functions. Amongst the functions tested was dot product. Using the dot product, the other parameters that were selected in the optimization process pointed to a unified weight for all components in the mixtures. That is equivalent to a dot product of the sum of vectors. That is, the data pointed to a dot product of sums of vectors as a good model. Once led to a dot product of a sum of vectors, normalizing by the size of the vectors was also needed to eliminate the effect of the sheer number of components in a mixture. At this point pairwise distance was already very close to an angle distance metric, after all, the cosine of the angle is the normalized dot product. When finally arriving at an angle distance model the results were consistent with the comparisons of identical mixtures and the correlation was much stronger even without any added parameters.

Consistency with Behavior and Neurobiology

In simple terms, the superior performance of the angle-distance model over the pairwise-distance model suggests a system that does not consider each mixture component alone, but rather a system that, through some configurational process, represents the mixture as a whole. This is in fact highly consistent with olfactory behavior and neural representation. In behavior, humans are very poor at identifying components in a mixture, even when they are highly familiar with the components alone. The typical maximum number of equal-intensity components humans can identify in a mixture is four. The number is independent of odorant type, and does not change even with explicit training. Moreover, perceptual features associated with a mono-molecule may sometimes make their way into a mixture containing that molecule, but sometimes not, and the rules for this remain unknown. In other words, like the present algorithm, human perception groups many mono-molecular components into singular unified percepts. This pattern, referred to as either associative, synthetic, or configural, is in contrast to the alternative of retaining individual mixture component identity, referred to as dissociative, analytical, or elemental. Although these patterns are not mutually exclusive, evidence from perception points to a primarily configural process in olfaction. Mixture synthesis may begin with a balance of agonistic and antagonistic interactions between mono-molecules at olfactory receptors in the epithelium or at glomeruli in the olfactory bulb. Thus, when components compete for common receptors, they may be harder to pick out of the mixture. The configural mechanisms in epithelium and bulb are further reflected in the cortex where patterns of neural activity induced by a mixture are unique, and not a combination of neural activity induced by the mixtures' components alone. In other words, like the present algorithm, the olfactory system at the neural level treats odorant-mixtures as unitary synthetic objects, and not as an analytical combination of components.

### Further Optimization of the Model

Although the model as described above performs well, it has three notable limitations. The first is that the mixtures studied were made of components that were first individually diluted to a point of equal perceived intensity. Intensity influences olfactory perception in complex ways, and some odorants, such as indole, can sharply shift in percept with changing intensity. Moreover, whereas some odorants can increase the overall intensity of a mixture they are added to, other odorants can reduce overall mixture intensity. Given this complexity, one may assume that when one of two mixtures under comparison contains intensity-sensitive molecules such as indole, the power of the present model may diminish. Notably, the independent test of the present model (FIG. 9E, 9F) implies that a perceived equality of intensity may not be a condition for the model to apply in the case of mono-molecular odorants. That said, the model may break down in mixtures whose components have not been at all equated for perceived intensity. With this in mind, a further optimization of the model incorporates optimizations for the prediction of odorant detection threshold as a proxy for intensity. These models may provide an intensity coefficient that may allow applying the present model to mixtures made of components that were not first equated for intensity.

A limitation is related to the odorants used for model building and testing. If the odorants represent only a limited portion of olfactory perceptual space, then the present model may apply to this portion of olfactory space alone. To protect against this, the present model uses the largest datasets available in order to build the model, and has been tested against subsets of the data not included in model building.

A similar limitation is in the selection of physicochemical features. Again, the more features one incorporates into a model, the smaller the risk of not capturing the relevant sources of variance, and the present model thus includes more than a thousand features.

Thus, the present embodiments may provide an algorithm that allows predicting odorant-mixture perceptual similarity from odorant-mixture structure. The synthetic nature of the algorithm is consistent with the synthetic nature of olfactory perception and neural representation. Such an algorithm may further serve as a framework for theory-based selection of components for odorant-mixtures in studies of olfactory processing.

### Methods

### Subjects

We tested 139 normosmic and generally healthy subjects, of whom 63 were women, and all were between the ages of 21 and 45.

### General Procedures

The experiments were conducted in stainless-steel-coated rooms with HEPA and carbon filtration designed to minimize olfactory contamination. All interactions with subjects during experiments were by computer, and subjects provided their responses through a computer keyboard or mouse. Odorant mixtures were sniffed from jars marked arbitrarily, and presentation order was counterbalanced across subjects. In order to minimize olfactory adaptation, a −40 second inter-trial interval was maintained between presentations.

### Equated-Intensity Odorants

All odorants were purchased or otherwise obtained at the highest available purity. All odorants were diluted with either mineral oil, 1,2-propanediol or deionized distilled water to a point of approximately equally perceived intensity. The perceived-intensity equation was conducted according to previously published methods [29]. In brief, we identified the odorant with lowest perceived intensity, and first diluted all others to equal perceived intensity as estimated by experienced lab members. Next, 24 naive subjects, including 10 females, smelled the odorants, and rated their intensity. We then further diluted any odorant that was 2 or more standard deviations away from the mean intensity of the series, and repeated the process until we had no outliers. This process is suboptimal, but considering the natural variability in intensity perception, together with naive subjects' bias to identify a difference, and the iterative nature of this procedure, any stricter criteria would generate an endless process.

### GCMS Verification

To verify that the present method of odorant-mixture preparation and delivery did not generate novel compounds, one set of mixtures (Dataset #2) was analyzed with GCMS. In brief, the experimenters left the samples to sit in closed vials for several hours, then incubated over night at 50° C. This was done to accelerate the kinetics of any potential reactions that may have occurred. All the individual components (mono-molecules) of the mixtures were run separately, to ascertain their purity. The single peak retention times and corresponding spectrum identifications were noted and verified using Wiley Registry 9^{th} Edition/NIST 2008 combined mass spectral library (Wiley, New York, N.Y.). The mixture samples were then subjected to the same GCMS method as the single components, and Total Ion Chromatogram peaks were validated to contain only the expected peaks of their constituting single components. Peaks with wide or abnormal shapes were subjected to further spectrum deconvolution to assess potentially overlapping peaks. All analyses were made using a Gas Chromtograph coupled to a Mass Spectrometer, integrated with a headspace sampler. Prior to injection, samples were incubated in the agitator for 5 minutes under 35° C. and 250 rpm agitation. One ml of vial headspace gas was drawn into a heated syringe and injected to a split/splitless inlet that was kept at 250° C. and a Split ratio of 5:1. The GC method used a HP-5 MS column (30 m×0.25 mm×0.25 Jlm) and Helium as a carrier gas with 1.5 ml/min constant flow. Temperature program was 50° C. for 3 minutes, 15° C./min ramp up to 250° C. for 3 minutes. MS scans were conducted in Electron Impact mode (70 eV) from m/z 40 to 550, 2.86 scans/sec. MS source and Quad temperature were 230° C. and 150° C., respectively.

### Pairwise Similarity Tests

In each trial, each subject was presented with two mixtures and was asked to rate their similarity on a VAS. The question at the top of the VAS was “To what extent are these two odors similar” and the VAS scale ranged from “not at all” to “highly”. In Data-Set #1 the VAS was also numerated from 1 (“not at all”) to 9 (“very”), and in the remaining data-sets it was not numerated. In both cases, the ratings were normalized within subjects to a scale of 0% to 100%. Each subject repeated the experiment on two different days to assess test-retest reliability. An arbitrary cutoff applied whereby if the difference between 2 repetitions of the same comparison was greater than 70%, the rating was excluded. This amounted to 109 out of 2070 ratings (−5%) in Dataset #1, and no deletions in Datasets #2 and #3. The ratings by subjects whose similarity ratings for identical mixtures were poorer by at least 2 standard deviations from the mean were discarded. This amounted to 3 subjects. The average rated similarities were calculated across subjects.

TABLE 1
List of 21 descriptors for optimized mixture similarity prediction Listed
are the names, indices and a brief definition of the 21 descriptors selected as the
optimized set in our angle distance model for odorant mixture similarity prediction.

| | | Index out | |
|---|---|---|---|
| Description | Abbreviation | of 1433 | No. |
| Number of circuits (constitutional descriptors). | nCIR | 19 | 1 |
| First Zagreb index M1 (topological descriptors). | ZM1 | 44 | 2 |
| Nanuni geometric topological index | GNar | 51 | 3 |
| topological descriptors). | | | |
| 1-path Kier alpha-modified shape index | SIK | 96 | 4 |
| (topological descriptors). | | | |
| Molecular multiple path count of order 08 | piPC08 | 175 | 5 |
| (walk and path counts). | | | |
| Moran autocorrelation-lag 1 I weighted by | MATS1v | 289 | 6 |
| atomic van der | | | |
| Moran autocorrelation-lag 7 I weighted | MATS7v | 295 | 7 |
| by atomic van der | | | |
| Geary autocorrelation-lag 1 I weighted | GATS1v | 321 | 8 |
| by atomic van der | | | |
| Eigenvalue 05 from edge adj. Matrix | EEig05x | 351 | 9 |
| weighted by edge degrees | | | |
| Spectral moment 02 from edge adj. Matrix | ESpm02x | 407 | 10 |
| weighted by edge degrees (edge adjacency indices). | | | |
| Spectral moment 03 from edge adj. matrix weighted | ESpm03d | 423 | 11 |
| by dipole moments (edge adjacency indices). | | | |
| Spectral moment 10 from edge adj. matrix weighted | ESpm10d | 430 | 12 |
| by dipole moments (edge adjacency indices). | | | |
| Spectral moment 13 from edge adj. matrix weighted | ESpm13d | 433 | 13 |
| by dipole moments (edge adjacency indices). | | | |
| Lowest eigenvalue n. 3 of Burden matrix I weighted | BELv3 | 477 | 14 |
| by atomic | | | |
| Radial Distribution Function-3.5 I weighted by | RDF035v | 733 | 15 |
| atomic van der | | | |
| 1⁵ component symmetry directional WHIM | G1m | 994 | 16 |
| index I weighted by | | | |
| 1⁵ component symmetry directional | G1v | 1005 | 17 |
| index I weighted by | | | |
| 1⁵ component symmetry directional WHIM | G1e | 1016 | 18 |
| index I weighted by | | | |
| 3′ component symmetry directional WHIM | G3s | 1040 | 19 |
| index I weighted by | | | |
| R maximal autocorrelation of lag 8 I | R8u+ | 1200 | 20 |
| unweighted (GETAWAY | | | |
| Number of thioesters (aliphatic) | nRCOSR | 1295 | 21 |
| (Functional group counts) | | | |

Datasets: The following table contains the average normalized similarity rating applied to each comparison, by dataset. The fourth list of CID numbers is from Wright and Michels (1964).

Dataset #1
Dataset #1 comparisons

| Comparison | Mixture | Mixture | Average rated |
|---|---|---|---|
| number | Number | Number | similarity |
| 1 | 1 | 2 | 39.5833333333 |
| 2 | 1 | 3 | 34.8958333333 |
| 3 | 1 | 4 | 47.3958233223 |
| 4 | 1 | 5 | 49.4791866667 |
| 5 | 1 | 6 | 58.8541666667 |
| 6 | 1 | 7 | 43.75 |
| 7 | 8 | 2 | 24.4791666667 |
| 8 | 8 | 3 | 31.5104166667 |
| 9 | 8 | 4 | 15.1041666667 |
| 10 | 8 | 5 | 23.4375 |
| 11 | 8 | 3 | 19.2708333333 |
| 12 | 8 | 7 | 9.8958333333 |
| 13 | 9 | 2 | 43.2291666667 |
| 14 | 9 | 3 | 32.8125 |
| 15 | 9 | 4 | 57.5520833333 |
| 16 | 9 | 5 | 60.9375 |
| 17 | 9 | 6 | 55.2082333323 |
| 18 | 9 | 7 | 38.0208333333 |
| 19 | 10 | 2 | 43.2291666667 |
| 20 | 10 | 3 | 34.8958333333 |
| 21 | 10 | 4 | 45.8333333333 |
| 22 | 10 | 5 | 63.0208333333 |
| 23 | 10 | 6 | 58.8541666667 |
| 24 | 10 | 7 | 54.1666666667 |
| 25 | 11 | 2 | 48.9583333333 |
| 26 | 11 | 3 | 28.6458333333 |
| 27 | 11 | 4 | 53.125 |
| 28 | 11 | 5 | 65.625 |
| 29 | 11 | 6 | 61.9791666667 |
| 30 | 11 | 7 | 44.7916666667 |
| 31 | 12 | 2 | 22.9166666667 |
| 32 | 12 | 3 | 23.4375 |
| 33 | 12 | 4 | 30.2083333333 |
| 34 | 12 | 5 | 31.7708333333 |
| 35 | 12 | 6 | 36.9791666667 |
| 36 | 12 | 7 | 28.90625 |
| 37 | 13 | 14 | 24.5192307692 |
| 38 | 13 | 15 | 29.8076923077 |
| 39 | 13 | 16 | 29.3269230769 |
| 40 | 13 | 17 | 41.8269230769 |
| 41 | 13 | 18 | 43.2692307692 |
| 42 | 13 | 19 | 17.7884615385 |
| 43 | 20 | 14 | 28.8461538462 |
| 44 | 20 | 15 | 46.6346153846 |
| 45 | 20 | 16 | 24.5192307692 |
| 46 | 20 | 17 | 22.5961538462 |
| 47 | 20 | 18 | 27.8846153846 |
| 48 | 20 | 19 | 46.6346153846 |
| 49 | 21 | 14 | 26.4423076923 |
| 50 | 21 | 15 | 28.8461538462 |
| 51 | 21 | 16 | 42.7884615385 |
| 52 | 21 | 17 | 48.5576923077 |
| 53 | 21 | 18 | 46.6346153846 |
| 54 | 21 | 19 | 31.7307692308 |
| 55 | 22 | 14 | 26.4423076923 |
| 56 | 22 | 15 | 31.7307692308 |
| 57 | 22 | 16 | 54.8076923077 |
| 58 | 22 | 17 | 57.2115384615 |
| 59 | 22 | 18 | 50 |
| 60 | 22 | 19 | 20.6730769231 |
| 61 | 23 | 14 | 24.5192307692 |
| 62 | 23 | 15 | 32.6923076923 |
| 63 | 23 | 16 | 50 |
| 64 | 23 | 17 | 54.8076923077 |
| 65 | 23 | 18 | 58.1730769231 |
| 66 | 23 | 19 | 22.1153846154 |
| 67 | 24 | 14 | 22.1153846154 |
| 68 | 24 | 15 | 29.8076923077 |
| 69 | 24 | 16 | 26.4423076923 |
| 70 | 24 | 17 | 25.4807692308 |
| 71 | 24 | 18 | 22.1153846154 |
| 72 | 24 | 19 | 32.2115384615 |
| 73 | 25 | 26 | 28.8461538462 |
| 74 | 25 | 27 | 27.8846153846 |
| 75 | 25 | 28 | 37.0192307692 |
| 76 | 25 | 29 | 32.6923076923 |
| 77 | 25 | 30 | 33.6538461538 |
| 78 | 25 | 31 | 38.9423076923 |
| 79 | 32 | 26 | 18.75 |
| 80 | 32 | 27 | 27.8846153846 |
| 81 | 32 | 28 | 20.6730769231 |
| 82 | 32 | 29 | 38.9423076923 |
| 83 | 32 | 30 | 25.9615384615 |
| 84 | 32 | 31 | 24.5192307692 |
| 85 | 33 | 26 | 31.7307692308 |
| 86 | 33 | 27 | 38.4615384615 |
| 87 | 33 | 28 | 26.4423076923 |
| 88 | 33 | 29 | 46.6346153846 |
| 89 | 33 | 30 | 48.0769230769 |
| 90 | 33 | 31 | 27.4038461538 |
| 91 | 34 | 26 | 34.1346153846 |
| 92 | 34 | 27 | 36.5384615385 |
| 93 | 34 | 28 | 30.7692307692 |
| 94 | 34 | 29 | 47.5961538462 |
| 95 | 34 | 30 | 54.3269230769 |
| 96 | 34 | 31 | 30.7692307692 |
| 97 | 35 | 26 | 26.4423076923 |
| 98 | 35 | 27 | 34.6153846154 |
| 99 | 35 | 28 | 32.6923076923 |
| 100 | 35 | 29 | 37.0192307692 |
| 101 | 35 | 30 | 48.5576923077 |
| 102 | 35 | 31 | 34.6153846154 |
| 103 | 36 | 26 | 23.0769230769 |
| 104 | 36 | 27 | 34.6153846154 |
| 105 | 36 | 28 | 28.3653846154 |
| 106 | 36 | 29 | 19.2307692308 |
| 107 | 36 | 30 | 23.5576923077 |
| 108 | 36 | 31 | 17.3076923077 |
| 109 | 37 | 38 | 47.7272727273 |
| 110 | 37 | 39 | 37.5 |
| 111 | 37 | 40 | 35.7954545455 |
| 112 | 37 | 41 | 37.5 |
| 113 | 42 | 39 | 47.1590909091 |
| 114 | 42 | 40 | 46.0227272727 |
| 115 | 42 | 41 | 52.8409090909 |
| 116 | 3 | 38 | 22.1590909091 |
| 117 | 3 | 39 | 22.7272727273 |
| 118 | 3 | 40 | 27.2727272727 |
| 119 | 3 | 41 | 30.6818181818 |
| 120 | 43 | 44 | 34.0909090909 |
| 121 | 43 | 38 | 33.5227272727 |
| 122 | 43 | 45 | 15.9090909091 |
| 123 | 43 | 39 | 35.7954545455 |
| 124 | 43 | 40 | 34.6590909091 |
| 125 | 43 | 41 | 35.2272727273 |
| 126 | 43 | 46 | 31.25 |
| 127 | 47 | 44 | 33.5227272727 |
| 128 | 47 | 38 | 60.7954545455 |
| 129 | 47 | 45 | 21.0227272727 |
| 130 | 47 | 39 | 43.75 |
| 131 | 47 | 40 | 51.1363636364 |
| 132 | 47 | 41 | 46.5909090909 |
| 133 | 47 | 46 | 38.0681818182 |
| 134 | 48 | 44 | 32.3863636364 |
| 135 | 48 | 38 | 58.5227272727 |
| 136 | 48 | 45 | 24.4318181818 |
| 137 | 48 | 39 | 55.6818181818 |
| 138 | 48 | 40 | 65.3409090909 |
| 139 | 48 | 41 | 47.7272727273 |
| 140 | 48 | 46 | 47.1590909091 |
| 141 | 49 | 44 | 64.7727272727 |
| 142 | 49 | 38 | 54.5454545455 |
| 143 | 49 | 45 | 32.3863636364 |
| 144 | 49 | 38 | 38.0681818182 |
| 145 | 49 | 40 | 35.2272727273 |
| 146 | 49 | 41 | 40.9090909091 |
| 147 | 49 | 46 | 39.7727272727 |
| 148 | 8 | 8 | 95.3125 |
| 149 | 12 | 12 | 96.875 |
| 150 | 1 | 1 | 91.6666666667 |
| 151 | 9 | 9 | 91.6666666667 |
| 152 | 10 | 10 | 88.5416666667 |
| 153 | 11 | 11 | 85.4166666667 |
| 154 | 2 | 2 | 95.8333333333 |
| 155 | 3 | 3 | 95.8333333333 |
| 156 | 4 | 4 | 91.6666666667 |
| 157 | 5 | 5 | 87.5 |
| 158 | 6 | 6 | 95.8333333333 |
| 159 | 7 | 7 | 100 |
| 160 | 14 | 14 | 97.1153846154 |
| 161 | 15 | 15 | 93.2692307692 |
| 162 | 16 | 16 | 81.7307692308 |
| 163 | 17 | 17 | 87.5 |
| 164 | 18 | 18 | 87.5 |
| 165 | 19 | 19 | 81.7307692308 |
| 166 | 13 | 13 | 91.3461538462 |
| 167 | 20 | 20 | 91.3461538462 |
| 168 | 21 | 21 | 92.3076923077 |
| 169 | 22 | 22 | 91.3461538462 |
| 170 | 23 | 23 | 88.4615384615 |
| 171 | 24 | 24 | 94.2307692308 |
| 172 | 32 | 32 | 100 |
| 173 | 36 | 36 | 100 |
| 174 | 25 | 25 | 90.3846153846 |
| 175 | 33 | 33 | 94.2307692308 |
| 176 | 34 | 34 | 95.1923076923 |
| 177 | 35 | 35 | 82.6923076923 |
| 178 | 27 | 27 | 87.5 |
| 179 | 31 | 31 | 75 |
| 180 | 26 | 26 | 76.9230769231 |
| 181 | 26 | 26 | 90.3846153846 |
| 182 | 29 | 29 | 89.4230769231 |
| 183 | 30 | 30 | 90.3846153846 |
| 184 | 38 | 38 | 89.7727272727 |
| 185 | 39 | 39 | 71.5909090909 |
| 186 | 40 | 40 | 81.8181818182 |
| 187 | 41 | 41 | 86.3636363636 |
| 188 | 42 | 42 | 86.3636363636 |
| 189 | 43 | 43 | 76.1363636364 |
| 190 | 47 | 47 | 70.4545454545 |
| 191 | 48 | 48 | 79.5454545455 |

| Mixture number | Mixture Cids |
|---|---|
| 1 | [6501 264 2879 7685 7731 326 7888 61138 8030 1183] |
| 2 | [240 93009 323 8148 7762 3314 460 6184 798 6054] |
| 3 | [7710] |
| 4 | [31276 93009 11002 323 7966 8148 7632 22201 19310 7762 2758 3314 460 443158 |
| | 20859 7059 999 6544 7770 10430] |
| 5 | [10890 93009 11002 6982 323 8797 7966 8148 7632 31252 19310 7762 3314 460 |
| | 6184 8892 8103 12178 5281168 798 443158 20859 7059 91497 999 10821 6544 7770 |
| | 7714 10430] |
| 6 | [7710 31276 10890 240 93009 11002 6982 323 8797 7966 8148 24915 7632 22201 |
| | 31252 19310 7762 26331 2758 3314 460 8130 6184 8892 8103 12178 5281168 798 |
| | 443158 20859 7059 62444 91497 999 10821 6054 6544 7770 7714 10430] |
| 7 | [93009 460 443158 6544] |
| 8 | [5283349] |
| 9 | [7410 6501 264 5281515 6259976 307 7685 326 5283349 7749 7363 7888 7119 8635 |
| | 8918 6736 8030 5634 7921 1183] |
| 10 | [7410 6501 7600 7519 264 5281515 6259976 307 2879 7685 7731 326 5283349 7583 |
| | 7749 7363 8129 7888 61016 8635 8918 957 7991 61138 6654 8118 6736 10722 1140 |
| | 1183] |
| 11 | [7991 61138 6654 8118 6736 8030 6989 10722 1140 5634 7921 1183] |
| 12 | [7731 7749 7888 1183] |
| 13 | [22201 7749 460 61016 7119 61138 999 10821 6054 6544] |
| 14 | [323 7762 7363 7888 16666 8635 7059 7991 6736 8030] |
| 15 | [7059] |
| 16 | [10890 7519 323 7583 7762 26331 8892 7888 443158 16666 8635 91497 8918 957 |
| | 18827 8118 8030 6989 5634 10430] |
| 17 | [14286 31276 7600 7519 11002 6982 307 323 5283349 7762 26331 3314 7363 8892 |
| | 8103 7888 443158 16666 8635 7059 91497 957 18827 7770 6736 8030 6989 10722 |
| | 5634 10430] |
| 18 | [14286 7710 31276 10890 7600 7519 11002 6982 307 323 2879 7731 5283349 7583 |
| | 7762 26331 3314 7363 8892 8103 7888 443158 16666 8635 70559 62444 91497 8918 |
| | 957 18827 7991 7770 8118 6736 8030 6989 10722 5634 10430 7921] |
| 19 | [7731 8892 7888 7059] |
| 20 | [62336] |
| 21 | [6501 264 6259976 8797 7685 7632 22201 2758 460 8129 5281168 62336 798 61016 |
| | 20859 61138 10821 6054 1140 1183] |
| 22 | [6501 62433 264 5281515 7685 326 7966 8148 24915 7632 22201 31252 19310 2738 |
| | 8130 8129 6184 12178 798 61016 7119 20859 999 10821 6054 6544 6654 1140 7714 |
| | 1183] |
| 23 | [7410 6501 62433 240 93009 264 5281515 6259976 8797 7685 326 7966 8148 24915 |
| | 7632 22201 31232 19310 7749 2758 460 8130 8129 6184 12178 5281168 62336 798 |
| | 61016 7119 20859 61138 999 10821 6054 6544 6654 1140 7714 1183] |
| 24 | [7749 61138 6054 6544] |
| 25 | [7600 62433 307 5283349 443158 8635 8918 999 6736 10722] |
| 26 | [7410 10890 7519 7685 24915 26331 8129 16666 7770 10430] |
| 27 | [7714] |
| 28 | [7410 10890 93009 6259976 2879 8797 7685 24915 8103 5281168 7888 16666 18827 |
| | 7991 6054 6654 7770 8030 5634 10430] |
| 29 | [7410 10890 93009 11002 8797 7685 7731 7966 24915 7583 26331 5281168 7888 |
| | 798 61016 16666 7119 20859 18827 7991 6054 6654 7770 8118 8030 1140 7714 5634 |
| | 10430 1183] |
| 30 | [7410 10890 7519 93009 11002 6259976 323 2879 8797 7685 7731 326 7966 24915 |
| | 31252 7583 26331 460 8129 8103 5281168 7888 798 61016 16666 7119 20859 18827 |
| | 7991 10821 6054 6654 7770 8118 8030 1140 7714 5634 10430 1183] |
| 31 | [5281168 10890 2879 7966] |
| 32 | [31276] |
| 33 | [14286 7710 31276 7600 5281515 6982 5283349 7632 7762 3314 6184 443158 91497 |
| | 8918 957 61138 999 6544 6989 7921] |
| 34 | [14286 7710 31276 7600 62433 240 264 5281515 6982 307 5283349 7632 22201 |
| | 19310 7749 2758 3314 6184 8892 443158 7059 91497 8918 957 61138 6544 6736 |
| | 6989 10722 7921] |
| 35 | [14286 6501 7710 31276 7600 62433 240 264 5281515 6982 307 5283349 8148 7632 |
| | 22201 19310 7762 7749 2758 3314 7363 8130 6184 8892 12178 62336 443158 8635 |
| | 7059 62444 91497 8918 957 61138 999 6544 6736 6989 10722 7921] |
| 36 | [62433 7363 443158 61138] |
| 37 | [14286 7600 3314 16666 91497 18827 7991 7770 6989 5634] |
| 38 | [61199 10890 93009 264 6259976 24915 7762 460 8129 5281168 443158 8918 957 |
| | 999 17100] |
| 39 | [61199 6501 264 2879 7731 326 24915 7762 460 8129 8892 12178 5281168 443158 |
| | 4133 999 10821 17100 11552 1183] |
| 40 | [61199 6501 31276 10890 7519 240 93009 11002 6259976 2879 7685 7731 326 |
| | 5283349 7583 460 8129 8892 12178 443158 4133 8918 957 61138 999 10821 6544 |
| | 17100 11552 1183] |
| 41 | [61199 6501 31276 10890 7519 240 93009 264 11002 6259976 2879 8797 7685 7731 |
| | 326 7966 5283349 9609 24915 7583 7762 26331 460 8129 8892 12178 5281168 |
| | 443158 20859 4133 62444 8918 957 61138 999 10821 6544 6736 17100 31277 10722 |
| | 11552 1183] |
| 42 | [7410 7710 7600 62433 307 7749 3314 61016 16666 91497 6054 6654 8030 5634 |
| | 10430] |
| 43 | [7410 14286 7710 7600 62433 5281515 6982 22201 7749 3314 8130 8103 7888 |
| | 16666 91497 6654 7770 5634 10430 7921] |
| 44 | [6501 8797 326 26331 8129 12178 999 6544 6736 11552] |
| 45 | [7685] |
| 46 | [6501 460 999 6544] |
| 47 | [7410 7710 7600 62433 5281515 6982 307 7632 22201 31252 19310 7749 3314 6184 |
| | 7888 61016 16666 7119 91497 18827 7991 6054 6654 7770 8118 8030 6989 7714 |
| | 5634 10430] |
| 48 | [7410 14286 7710 7600 62433 5281515 6982 307 323 8148 7632 22201 31252 19310 |
| | 7749 2758 3314 7363 8130 6184 8103 62336 7888 798 61016 16666 7119 8635 7059 |
| | 91497 18827 7991 6054 6654 7770 8118 8030 6989 1140 7714 5634 10430 7921] |
| 49 | [7600 62433 7991 6989] |

Dataset #2
Dataset #2 comparisons

| Comparison | Mixture | Mixture | Average rated |
|---|---|---|---|
| number | Number | Number | similarity |
| 1 | 1 | 2 | 42.8920768277 |
| 2 | 1 | 3 | 38.2925188853 |
| 3 | 1 | 4 | 58.2205435883 |
| 4 | 5 | 6 | 29.7321081182 |
| 5 | 5 | 7 | 62.231981175 |
| 6 | 5 | 3 | 59.6834225837 |
| 7 | 2 | 5 | 56.8320625991 |
| 8 | 2 | 6 | 31.1102534239 |
| 9 | 8 | 2 | 45.1906525188 |
| 10 | 8 | 9 | 55.8460436439 |
| 11 | 6 | 7 | 27.1032905381 |
| 12 | 6 | 10 | 28.4666081119 |
| 13 | 6 | 11 | 37.8212120261 |
| 14 | 12 | 5 | 29.2254453261 |
| 15 | 12 | 2 | 32.8488419076 |
| 16 | 12 | 6 | 35.9348363339 |
| 17 | 12 | 10 | 37.0957060269 |
| 18 | 7 | 1 | 35.8676065026 |
| 19 | 7 | 8 | 38.8315476659 |
| 20 | 3 | 12 | 29.3431840677 |
| 21 | 3 | 13 | 41.8740722418 |
| 22 | 3 | 4 | 55.1835934311 |
| 23 | 3 | 10 | 44.6881379562 |
| 24 | 9 | 5 | 61.8433647714 |
| 25 | 9 | 12 | 30.0817078966 |
| 26 | 9 | 3 | 49.1864076834 |
| 27 | 9 | 14 | 54.434006142 |
| 28 | 13 | 1 | 45.0479865702 |
| 29 | 13 | 2 | 43.3056175159 |
| 30 | 13 | 6 | 40.0733972789 |
| 31 | 4 | 5 | 71.0763747141 |
| 32 | 4 | 8 | 51.6250918479 |
| 33 | 4 | 13 | 37.7755842727 |
| 34 | 4 | 11 | 42.65543746 |
| 35 | 10 | 9 | 51.6787465177 |
| 36 | 10 | 4 | 60.041397948 |
| 37 | 14 | 1 | 34.334684991 |
| 38 | 14 | 6 | 33.6834812847 |
| 39 | 14 | 7 | 66.8014539949 |
| 40 | 14 | 13 | 40.4904882931 |
| 41 | 14 | 10 | 65.2906207311 |
| 42 | 11 | 1 | 62.0149033493 |
| 43 | 11 | 2 | 52.1849505052 |
| 44 | 11 | 8 | 48.0076235013 |
| 45 | 11 | 12 | 34.7939733695 |
| 46 | 11 | 13 | 50.4446400068 |
| 47 | 1 | 5 | 63.4176348598 |
| 48 | 1 | 8 | 35.9579997488 |
| 49 | 1 | 6 | 44.5168647674 |
| 50 | 1 | 12 | 53.8750343555 |
| 51 | 1 | 9 | 46.8743338229 |
| 52 | 1 | 10 | 37.0116310677 |
| 53 | 5 | 8 | 47.6427082577 |
| 54 | 5 | 13 | 37.6277234001 |
| 55 | 5 | 10 | 47.5206029328 |
| 56 | 5 | 14 | 56.5273711569 |
| 57 | 5 | 11 | 55.5547834727 |
| 58 | 2 | 7 | 56.5124839064 |
| 59 | 2 | 3 | 47.8892521298 |
| 60 | 2 | 9 | 56.4702011828 |
| 61 | 2 | 4 | 61.0520828953 |
| 62 | 2 | 10 | 59.0501557976 |
| 63 | 2 | 14 | 64.6282394837 |
| 64 | 8 | 6 | 30.0333647715 |
| 65 | 8 | 12 | 24.9943769886 |
| 66 | 8 | 3 | 50.605626467 |
| 67 | 8 | 13 | 23.3561339388 |
| 68 | 8 | 10 | 46.2247464518 |
| 69 | 8 | 14 | 38.2099169932 |
| 70 | 6 | 3 | 35.1094674536 |
| 71 | 6 | 9 | 27.793943301 |
| 72 | 6 | 4 | 28.1503345953 |
| 73 | 12 | 7 | 33.8501517588 |
| 74 | 12 | 13 | 36.6066038191 |
| 75 | 12 | 4 | 27.5310341851 |
| 76 | 12 | 14 | 39.1216385083 |
| 77 | 7 | 3 | 53.5510491156 |
| 78 | 7 | 9 | 58.2561770446 |
| 79 | 7 | 13 | 43.9005771667 |
| 80 | 7 | 4 | 61.4611468128 |
| 81 | 7 | 10 | 50.0969153042 |
| 82 | 7 | 11 | 65.5970916721 |
| 83 | 3 | 14 | 48.393467523 |
| 84 | 3 | 11 | 50.3668346769 |
| 85 | 9 | 13 | 41.7041072969 |
| 86 | 9 | 4 | 58.5990436446 |
| 87 | 9 | 11 | 69.0992488397 |
| 88 | 13 | 10 | 39.051042677 |
| 89 | 4 | 14 | 63.0563164143 |
| 90 | 10 | 11 | 45.9789168529 |
| 91 | 14 | 11 | 62.5123193783 |
| 92 | 1 | 1 | 70.2901207791 |
| 93 | 5 | 5 | 58.2890207475 |
| 94 | 11 | 11 | 69.6266983069 |
| 95 | 14 | 14 | 68.4690574039 |

| Mixture number | Mixture Cids |
|---|---|
| 1 | [326 26331 6544 1140] |
| 2 | [7710 62433 7519 7685 3314] |
| 3 | [31276 62433 7519 8129 12178 18827 10722] |
| 4 | [62433 8797 2758 3314 8635 61138 6054 6544 10722] |
| 5 | [7410 240 93009 8635] |
| 6 | [7519 8148 31252 8103 5281168 6544] |
| 7 | [240 307 7731 2758 12178 62336 8635] |
| 8 | [31276 8148 7762 18827 7714] |
| 9 | [7710 93009 8130 8103 5281168 7059 8918 7714] |
| 10 | [11002 307 7685 12178 4133 7991 6054 7770 7714] |
| 11 | [240 2758 8130 8129 5281168 7059 4133 8918 957 6654] |
| 12 | [7410 326 2758 62444 7770 1140] |
| 13 | [7410 7519 11002 8797 8129 5281168 6654 8030] |
| 14 | [8797 7731 7966 3314 62336 7059 7991 61138 6064 6544] |

Dataset #3
Dataset #3 comparisions

| Comparison | | | Average rated |
|---|---|---|---|
| number | CID | CID | Similarity |
| 1 | 7410 | 19310 | 14.6836842105 |
| 2 | 7710 | 7749 | 30.4985 |
| 3 | 31276 | 3314 | 42.0935 |
| 4 | 7519 | 8129 | 48.2145 |
| 5 | 240 | 8103 | 59.6205 |
| 6 | 93009 | 12178 | 48.0875 |
| 7 | 11002 | 62336 | 34.136 |
| 8 | 7685 | 8635 | 51.213 |
| 9 | 7731 | 62444 | 11.8755 |
| 10 | 326 | 8918 | 53.1495 |
| 11 | 8148 | 7991 | 49.2995 |
| 12 | 9609 | 61138 | 52.0752631579 |
| 13 | 22201 | 1140 | 15.3265 |
| 14 | 31252 | 10430 | 27.067 |
| 15 | 31276 | 26331 | 21.4161181775 |
| 16 | 6054 | 31276 | 47.2128292008 |
| 17 | 240 | 326 | 40.274739359 |
| 18 | 93009 | 240 | 52.9339534823 |
| 19 | 7685 | 7762 | 33.6845511624 |
| 20 | 8148 | 93009 | 13.9985159777 |
| 21 | 7762 | 8129 | 39.8168634983 |
| 22 | 7749 | 7519 | 63.3664229027 |
| 23 | 26331 | 8148 | 41.4196129139 |
| 24 | 3314 | 11002 | 20.7514735389 |
| 25 | 62336 | 22201 | 15.9907826016 |
| 26 | 7059 | 7685 | 59.2873712665 |
| 27 | 4133 | 31252 | 32.2346601009 |
| 28 | 8030 | 62336 | 30.1873834883 |
| 29 | 7519 | 8030 | 34.9673839804 |
| 30 | 326 | 7059 | 50.4071198260 |
| 31 | 22201 | 7714 | 15.6632745813 |
| 32 | 31252 | 6054 | 37.5950057271 |
| 33 | 8129 | 4133 | 48.3947495328 |
| 34 | 6654 | 7714 | 31.1489998884 |
| 35 | 7410 | 3314 | 40.218635755 |
| 36 | 7410 | 12178 | 53.7903638806 |
| 37 | 7710 | 307 | 49.0692920675 |
| 38 | 7710 | 8130 | 31.8349010241 |
| 39 | 61138 | 7410 | 18.3113202284 |
| 40 | 10821 | 7710 | 41.5299748449 |
| 41 | 6544 | 31276 | 37.1413111883 |
| 42 | 8797 | 8130 | 49.9228372081 |
| 43 | 7731 | 8797 | 51.5322298853 |
| 44 | 8103 | 8148 | 22.6170029804 |
| 45 | 12178 | 62433 | 43.1011214638 |
| 46 | 5261168 | 2758 | 36.8672625169 |
| 47 | 5281168 | 8103 | 56.9387376195 |
| 48 | 62444 | 240 | 9.8495700282 |
| 49 | 957 | 8148 | 22.6042669165 |
| 50 | 957 | 8129 | 86.6011726073 |
| 51 | 18827 | 93009 | 30.1616686586 |
| 52 | 7991 | 2758 | 16.6954798309 |
| 53 | 10821 | 7731 | 51.3415861985 |
| 54 | 6544 | 5281168 | 53.6374800249 |
| 55 | 7770 | 307 | 42.8196854198 |
| 56 | 8118 | 240 | 29.1133737842 |
| 57 | 8118 | 11002 | 74.7306409532 |
| 58 | 62433 | 957 | 57.6383061725 |
| 59 | 62433 | 8030 | 21.7308202652 |
| 60 | 10722 | 7731 | 55.9731866722 |
| 61 | 1140 | 26331 | 29.1266581311 |
| 62 | 307 | 7991 | 9.3496150027 |
| 63 | 8797 | 10821 | 48.8879859743 |
| 64 | 2758 | 10722 | 57.8195838198 |
| 65 | 8129 | 5054 | 46.3080427806 |
| 66 | 8129 | 7770 | 40.35004591 |
| 67 | 8103 | 8918 | 49.638579144 |
| 68 | 12178 | 7714 | 36.3096194455 |
| 69 | 62444 | 1140 | 14.8323293991 |
| 70 | 8918 | 7059 | 50.351401804 |
| 71 | 8918 | 61138 | 27.6837704748 |
| 72 | 7991 | 10722 | 19.1925053919 |
| 73 | 61138 | 6054 | 26.0335811646 |
| 74 | 7770 | 6544 | 44.4479882844 |

Wright & Michels-Dataset CID's
CID

7888
17100
637566
8842
8184
8174
8914
263
1031
702
5943
638011
22311
6448
241
8078
9253
8079
8882
180
1254
637511
1032
176
996
2969
264
16590
402
6736
1049
7222
7969

The terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment, and the above description is to be construed as if this combination were explicitly written. Conversely, various features of the invention, which are, for brevity, described in the context of a single embodiment, may also be provided separately or in any suitable subcombination or as suitable in any other described embodiment of the invention, and the above description is to be construed as if these separate embodiments were explicitly written. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for comparing odors comprising:
sampling a first odor source and detecting primary odorants of said first odor source;
sampling a second odor source and detecting primary odorants of said second source;
for each odor source, storing each of the sampled odor sources in respective primary vectors of odor descriptors;
for each source respectively building a source vector of detected primary odorants by summing said primary vectors of the respectively detected primary odorants;
determining an angle between said first and second source vectors; and
outputting said determined angle as a comparison between said first and second odor sources.

2. The method of claim 1, comprising determining said angle from a dot product calculated between said source vectors.

3. The method of claim 2, comprising determining said angle by normalizing said dot product, said normalizing comprising dividing said dot product by a multiple of norms of said source vectors to obtain a normalized ratio.

4. The method of claim 3, comprising obtaining said angle by applying an inverse cosine operation to said normalized ratio.

5. The method of claim 1, wherein said descriptors making up said primary vectors are constructed from a set of physicochemical odor descriptors.

6. The method of claim 5, comprising obtaining an initially relatively large set of said physicochemical descriptors and carrying out dimension reduction by retaining ones of said of physicochemical descriptors shown experimentally to contribute by more than an average to a final comparison result.

7. The method of claim 6, wherein said initially relatively large set comprises is in excess of a thousand of said of physicochemical descriptors of which a set of twenty is retained following said dimension reduction, such that said component vectors have a dimension of twenty.

8. The method of claim 1, comprising normalizing the respective source vectors.

9. An electronic nose device for detecting and comparing odors, comprising:
a sampling unit configured to sample odor sources and detect primary odorants therein;
a vectorising unit for configured to store each of the sampled odor sources as respective primary vectors, the primary vectors each defining one of said detected primary odorants in terms of a predetermined set of odor descriptors;
a summation unit configured to build a source vector for each detected odor source by summing said respective primary vectors and normalizing;
an odor comparison unit, configured to compare two detected odor sources by determining an angle between respective source vectors.

10. The electronic nose of claim 9, configured to determine said angle from a dot product calculated between said source vectors.

11. The electronic nose of claim 10, configured to determine said angle by normalizing said dot product, said normalizing comprising dividing said dot product by a multiple of norms of said source vectors to obtain a normalized ratio.

12. The electronic nose of claim 11, configured to obtain said angle by applying an inverse cosine operation to said normalized ratio.

13. The electronic nose of claim 9, wherein said descriptors making up said primary vectors are constructed from a set of physicochemical odor descriptors.
